(19) United States
(12) Patent Application Publication
(10) Pub. No.: US 2003/0087414 A1
(43) Pub. Date: May 8, 2003

(54) Mammalian mucinase, its recombinant production, and its use in therapy or prophylaxis against diseases in which mucus is involved or infectious diseases

(76) Inventors: Johannes Maria Franciscus Gerardus Aerts, Abcoude (NL); Rolf Gabriel Boot, Amsterdam (NL)

Correspondence Address: TRASK, BRITT & ROSSA, P.O. Box 2550, Salt Lake City, UT, 84110, US

(21) Appl. No.: 10/004,219

(22) Filed: Nov. 2, 2001

(51) Int. Cl. A61K 38/54; A61K 38/48; C12N 9/64

(52) U.S. Cl. 435/226; 424/94.63; 424/94.2

(57) ABSTRACT

The invention provides a mammalian mucinase capable of hydrolyzing mucin. Said mucinase is among others suitable for counteracting diseases in which mucus is involved. Said diseases comprise cystic fibrosis, COPD, asthma, bronchitis, tuberculosis, tumours with altered mucus expression, and mucus-containing pathogens. The invention also provides a pharmaceutical composition comprising an effective amount of said mucinase and a method of therapeutic or prophylactic treatment of an individual against a disease in which mucus is involved. Methods for obtaining said mucinase are also herewith provided, as well as nucleic acids encoding (part of) said mucinase. In one aspect the invention provides a diagnostic kit comprising a mucinase, a mucinase-specific antibody, a mucinase-derived peptide and/or nucleic acid encoding (part of) said mucinase.

### FIELD OF THE INVENTION

The invention relates to the field of medicine. More specifically, the invention relates to therapeutic or prophylactic treatment of an individual against a disease in which mucus is involved and/or an infection disease. The invention also relates to the preparation of a mucinase suitable for said treatment.

### BACKGROUND OF THE INVENTION

Mucus as Protective Barrier

Mucins form part of the dynamic, interactive defensive system of mammals at mucosal surfaces in for example the gastrointestinal tract, the respiratory tract, and reproductive organs. Mucins are highly glycosylated proteins occurring either as secretory or membrane bound forms. They have a unique molecular structure and chemical properties. The polypeptide backbone (apomucin) is rich in hydroxy amino acids, serine and threonine, which together with glycine, alanine and proline comprise nearly 50% of total amino acid residues of the protein, and are present as tandemly repeated sequences. The threonine and serine residues are the targets of O-glycosylation machinery and the extent of glycosylation is such that carbohydrates account for 50-85% of the dry weight of mucins. Secretory mucins are the major constituents of mucus secretions, lining the epithelial cells of digestive, respiratory and reproductive tracts (Gendler et al, 1995, Gum 1995). They are capable of forming gels at very low concentration by forming long thread like polymers resulting from the formation of disulphide linkages between monomers and intramolecular interactions of sugar side chains. Membrane bound mucins are present on the surface of various cell types and, unlike secretory mucins, do not form oligomers and are hence smaller in size than their secretory counterparts (Gendler et al, 1995, Gum 1995). The membrane bound mucins also have O-glycosylated serine and threonine rich regions, but they lack tandem repeat sequences. The primary function of secretory mucins is to provide protection to the apical epithelial cell layers in digestive, respiratory and urinogenital tracts against environmental factors like acidic pH, hydrolytic enzymes and pathogens. The cell surface mucins, in addition to their protective role, have shielding effect on various surface receptors, thereby helping in the regulation of their activity (Strous and Dekker 1992). So far, twelve human mucin genes have been identified designated as MUC1-4, MUC5AC, MUC5B, MUC6-9, and MUC11-12 (Gendler et al, 1995, Gum 1995, Gum et al 1990, Lan et al 1990, Moniaux et al 1999, Shankar et al 1997, Williams et al 1999). They can be divided into secreted and membrane-associated forms each with characteristic protein domains and tissue specific glycosylation. Eight human mucin genes have been well characterized: MUC2, MUC5AC, MUC5B, MUC6 map to 11p15.5 and encode secretory gel forming mucins while MUC1, MUC3, MUC4, MUC7 are scattered on different chromosomes and encode membrane-bound or secreted mucins. Historically, purified mucins have been identified by their amino and carbohydrate composition consisting of a high percentage of serine, threonine, proline, alanine, glycine, and a large proportion of O-linked oligosaccharides (up to 80% of the total mass). Biosynthetic pathways have been described for the secreted and membrane-associated mucins and their eventual degradation and turnover. Mucins are present at all mucosal surfaces throughout the body in typical combinations and relate to the demands of organ function. Patterns of MUC gene expression with gastrointestinal site specific glycosylation are clearly important but are not yet well defined. The mucosal surface throughout the gastrointestinal tract must resist the aggressive elements from the external environment present in the diet and encountered during normal function. This defensive system is based on fundamental characteristics shared with the barrier found at other mucosal surfaces. The stable protective barrier enabling exchange between the epithelial cells of the gut lumen for the purposes of nutrition and protection is made up of a layer of secreted mucus and a cell-surface membrane glycocalyx. The mucus defensive barrier forms the first line of defence to the external environment and contains both innate and adaptive immune elements.

Mucin Expression

Mucins are present at all mucosal surfaces throughout the body. The specific functional requirements for mucus at each site are reflected in the appearance of different mucins in the cells at different sites in the body. The main population of specially adapted cells producing secreted mucins are the goblet cells. The proportion of goblet cells increases through the gastrointestinal tract (GI tract) with maximal numbers in the rectum. Goblet cells in the respiratory tract are present in the trachea, and to a lesser extent in the bronchi. They are rarely found in bronchioles less than 1 mm in diameter (Jeffery et al 1992). Mucins have a tissue specific glycosylation at each site in the gastrointestinal tract. As carbohydrate constitutes the major part of all mature mucins and is represented by vast array of different oligosaccharide structures the potential for multiple functions related to bulk carbohydrate or individual structures must be examined. Most of the oligosaccharides in mucins are attached by O-links. However, a much smaller number of N-linked chains are also present, linked to asparagine residues in the mucin polypeptide through an N-glycosidic bond to N-acetyl-D-glucosamine. N-linked oligosaccharides contain a branched trimannosyl-chitobiose pentasaccharide core attached to the peptide.

Mucus Degradation

The adherent mucus barrier and the glycocalyx are constantly being turned over as part of their protective functions at the mucosal surface. Thus, degradation of mucus is a normal feature of an equilibrium between mucosal synthesis, secretion and the breakdown of the existing adherent gel. This balance must be regulated to ensure continual mucosal protection against potentially damaging compounds and organisms entering in the diet. The first stage in mucus degradation is conversion of the mucus gel to a viscous fluid. The viscoelastic properties of the secreted, adherent gel layer are governed by the mucins, they are responsible for the gel-forming properties. The non-mucin components of mucus may influence gel formation or affect gel strength. They are implicated in pathological situations where alterations in the normal composition of mucus are changed. Mucinase activity of bacteria has been described and is well known from observations of mucin carbohydrate release, sequestration and metabolic conversion by bacteria in the large intestine. A population of mucin oligosaccharide degrading (MOD) bacterial strains has been identified capable of specific and complete degradation of mucins. Bacterial mucinase enzymes have been shown to act on the mucus gel to reduce it to a viscous fluid, probably through the action of proteinases or peptidases, to act further on the accessible peptide backbone, not blocked by oligosaccharide substitution and to cleave the individual sugars from the oligosaccharide chains. Bacterial mucinase activity has been measured in faecal extracts using electrophoretic assessment of mucin degradation and by direct mucinase assays with purified biotinylated mucins.

Mucin-Associated Diseases

Changes in mucus are frequent in inflammatory diseases of the epithelia. High levels of secretion of the mucin proteins is a common factor in for instance cystic fibrosis (CF), chronic obstructive pulmonary disease (COPD), chronic bronchitis, asthma, tuberculosis, and carcinomas. Conversely, in Inflammatory Bowel Disease as Crohns disease and ulcerative colitis, the mucus barrier is decreased. Furthermore, mucus appears to play a role in infection by mucus-containing pathogens.

Mucus Obstruction in Cystic Fibrosis

Cystic Fibrosis is the most common lethal genetic disorder among Caucasians. Presently, approximately 30,000 children, adolescents, and adults in the United States are affected by this disease, as reported on the Cystic Fibrosis Foundation Web site (http://www.cff.org). The median age of survival is 32.3 years of age.

A variety of symptoms, the most common ones being salty-tasting sweat and skin, persistent cough, wheezing, and failure to thrive (due to intestinal defects, malnutrition and anorexia) characterize the disease. The usual complications in the respiratory tract are: hemoptysis (blood in the sputum); pneumothorax (collapsed lung); atelectasis (air resorbtion leaving the lobe or segment airless) caused by complete mucus plugging; dilated bronchioles and bronchi and weakened bronchioles and bronchi walls; fibrosis (scar tissue); and low oxygen levels. Respiratory failure in CF is usually at the end of a long process where frequently there is no longer enough healthy lung tissue left to eliminate CO₂ It is widely believed that the respiratory sequelae in CF and progressive deterioration of respiratory function are the result of persistent bacterial colonization (culminating with chronic P. aeruginosa infections) and chronic inflammation. The major cause of high morbidity and mortality in CF remain the chronic respiratory infections (most notably with with P. aeruginosa) which account for more than 90 percent of CF mortality.

Cystic fibrosis (CF) is a multiorgan disease that is the result of a genetic defect of a single gene. The gene, CF transmembrane conductance regulator (CFTR) was identified in 1989 (Riordan et al 1989). The gene encodes a membrane glycoprotein that functions as a cAMP-regulated chloride channel in exocrine glands and secretory epithelia.

As a result, thick and adhesive mucus is present in the airways and gastrointestinal tract of cystic fibrosis patients, leading to respiratory symptoms, recurrent infections, and progressive lung destruction, as well as nutritional deficiencies. The viscosity of CF mucus is determined by the presence of mucins. In addition, purulent mucus of CF patients contains as much as 3-14 mg/ml DNA (Chernick and Barbero 1959, Potter et al 1960). This DNA, derived from inflammatory cells and epithelial cells, contributes (together with actin) to the viscosity of purulent CF sputum. Therefore, mucus transport by mucociliary activity and/or cough is hampered. In addition, the viscous DNA containing mucus may also cause reduced effectiveness of aminoglycoside antibiotics. These two aspects of CF mucus viscosity result in persistent, recurring infections and progressive lung destruction.

As the pathogenesis of cystic fibrosis is complex, treatment for CF consists of several approaches. The pulmonary disease is managed by combinations of physiotherapy, antibiotics (especially to contain Pseudomonas aeruginosa infections, which is an important cause of death in CF patients), mucolytics (n-acetylcysteine, recombinant human DNAse I), bronchodilators and anti-inflammatory agents such as oral corticosteroids. The nutritional support mainly consists of the administration of pancreatic enzyme preparations to help food digestion, which is hampered by obstruction of the pancreatic ducts.

The aerosol route can be used to deliver mucoactive medications locally. These mucoactive medications comprise mucolytics, mucokinetic agents, mucoregulatory medications and expectorants and ion channel modifiers.

Several studies have focused on the use of recombinant human Dnase (rhuDNAse) I to reduce the viscosity of cystic fibrosis sputum. In in vitro assays the viscosity of purulent CF sputum as well as its adhesiveness was shown to decrease after treatment with recombinant human Dnase I (Pulmozyme, Genentech), and the mucociliary transportability of CF sputum is increased (Shak et al 1990 Zahm et al 1995,). This is due to the degradation of DNA but also to the depolymerization of F-actin (Vanscelllos et al 1994) Phase 3 clinical trials have shown that treatment with aerosolized recombinant human DNase I results in a 28-37% reduction in respiratory excacerbations and an improvement of 5.6-5.8% in FEV1, a measure for lung function (Fuchs et al 1994, Shak et al Chest 1995).

COPD

COPD is a physiologically defined group of conditions characterized by the presence of persistent airflow obstruction. COPD is defined as a disease state characterized by the presence of airflow obstruction due to chronic obstructive bronchitis or emphysema.

One of the hallmarks of bronchitis is hyperproduction of mucus as well as loss of mucociliary clearance. Even though an effect of rhuDNAse has been shown to reduce chronic bronchitis sputum viscosity (Puchelle et al 1996), mucus in chronic bronchitis contains 10-fold less DNA than in CF sputum (Kim et al 2001). This indicates that Pulmozyme, which is a human DNAse that is used to reduce viscosity of CF sputum, is not equally effective as a mucolytic for bronchitis mucus. Chronic bronchitis is currently treated with bronchodilators, β-adrenergic agents, methylxanthines, corticosteroids, and mucolytics, mostly with n-acetylcysteine N-acetylcystein has mucolytic activity in vitro, this activity has however not been demonstrated convincingly in vivo (Celli et al 1995)

Asthma

In chronic asthma, as in CF and bronchitis, decreased mucociliary clearance caused by mucus hypersecretion and/or rheological changes and permanent changes in ciliary structure and function occur. Airway inflammation plays a major role in the pathophysiology of asthma. Standard asthma treatment comprises antihistamines, bronchodilators, leukotrien inhibitors, and (gluco-)corticosteroids.

Tuberculosis

Another disease in which airway mucus viscosity is increased, and acetylcysteine is used as a mucolytic is pulmonary tuberculosis. Tuberculosis is an infectious disease caused by Mycobacterium tuberculosis, that is transmitted by aerosols of saliva and mucus released by coughing. Pulmonary tuberculosis is associated with persistent cough and expectoration of bloody mucus. The disease is generally treated by long-term therapy with combinations of antibiotics.

Intensive Care Medicine (Respiration)

Patients in intensive care that are attached to a respirator also have problems clearing airway mucus. The airways of these patients need to be cleared regularly to prevent stasis and oportunistic infections. To this aim these patients get antibiotics prophylactically.

Carcinoma

Mucins are thought to promote tumor-cell invasion and metastasis. In many human carcinomas, the expression profile of mucins is altered, with certain mucins like MUC1 being upregulated while others show a downregulated expression. The glycosylation process is disrupted in cancer, leading to aberrantly glycosylated, mostly underglycosylated mucins. In gastric carcinomas, the alterations of the mucin expression have been the Subject of several studies. The expression of MUC5AC, a secretory mucin present in normal gastric mucosa, is downregulated and can be found in only 60% of the intestinal carcinomas. At the same time the expression of MUC1 and MUC2 in gastric carcinomas was upregulated. The decrease in amount of glycosylation of MUC1 with the progression of carcinogenisis was shown with a panel of antibodies binding with different affinities to glycosylated and unglycosylated forms of MUC1. Furthermore, in cancer cells the expression of MUC1 was distributed over the entire cell membrane, while it was limited to the apical region of normal gastric mucosa cells.

The expression pattern of the mucin genes is complex in normal airways involving six genes, mainly MUC5AC and MUC5B in mucus-producing cells and MUC4 in a wide array of epithelial cells. MUC5AC overexpression in metaplasia, dysplasia and normal epithelium adjacent to squamous cell carcinoma provides additional arguments for a mucous cell origin of preneoplastic squamous lesions. MUC5AC and MUC5B expression is related to mucus formation in adenocarcinomas. Mucinous bronchioloalveolar carcinoma (BAC) has a particular pattern of mucin gene expression indicating that it has sustained a well-differentiated phenotype similar to the goblet cell, correlated with distinctive features i.e. a noninvasive pattern and a better prognosis than nonBACs. MUC4 is the earlier mucin gene expressed in the foregut, before epithelial differentiation and is expressed independently of mucus secretion both in normal adult airways and carcinomas. These findings are in favor of the histogenetic theory of non-small-cell carcinoma originating from a pluripotent mucous cell.

Several arguments suggest that mucins play a role in tumour-cell invasion and metastasis, resulting in prognostic implications. MUC1 is a transmembrane molecule with a large extracellular domain protruding high above the cell surface thought to reduce cell-cell and extracellular matrix (ECM)-cell adhesion in cancer cells (Jentoft 1990, Hudson et al 1996) but direct evidence for a role of specific mucin genes in tumor progression is lacking. One study shows that splenic-portal inoculation in athymic mice of MUC2 antisense construct in highly metastatic human colon cancer cells resulted in a reduction in MUC2 levels and a marked decrease in liver colonization (Sternberg et al 1999). Sialomucin complex (SMC), a rat homologue of the human mucin MUC4 isolated from highly metastatic ascites 13762 mammary adenocarcinoma cells is thought to potentiate metastasis by sterically disruption of molecular interactions for cell-cell and cell-ECM adhesions and by suppression of anti-tumor immunity by inhibition of interactions between cytotoxic lymphocytes and target tumor cells (Carraway et al 2000). One recent study shows that in vivo, subcutaneous injection of SMC-overexpressing cells results in substantially greater lung metastasis than injection of SMC-repressed cells. Moreover, injection of A375 human melanoma cells followed by in vivo induction of SMC overexpression within the solid tumor resulted in spontaneous distant metastasis (Komatsu et al 2000).

Mucus-Containing Pathogens

Mucins and mucin-like molecules have recently been described in several protozoan parasites, at different stages of the life cycle. These include kinetoplastid Trypanosoma, Leishmania), apicomplaxan (Cryptosporadium) and amoebic (Entamoeba) parasites (Schenkman et al 1993, Almeida et al 1994, Ilg et al 1999, Barnes et al 1998, Strong et al 2000). These share many structural and compositional features with mammalian mucins, but vary in several other aspects. It is now becoming evident that mucins in parasite are involved in cell-cell interaction and cell surface protection, thus helping the parasite to establish infection.

Currently, several pharmaceutical compounds against diseases in which mucus is involved are used. Those compounds have however limited beneficial effects. A major reason for this is the fact, that the targets of therapeutic compounds, like for instance lung epithelial cells, can hardly be reached because of the barrier of thick and adhesive mucus which is present in the airways and gastrointestinal tract of the patient. After administration of a certain pharmaceutical composition, only a small percentage of said composition is actually capable of performing its beneficial effect. Higher doses often do not improve treatment; essentially no more targets can be reached. Furthermore, higher doses often lead to more harmful side effects. A major part of administered pharmaceutical composition therefore often leaves the body before any beneficial effect could be performed.

The incapability of current therapeutic compounds to reach their target efficiently is a major drawback of current treatment.

Mucus thus provides an important defensive barrier which forms the first line of defence to the external environment. However, several diseases involve a disturbed generation of mucus, resulting in thick and adhesive mucus. This mucus forms an unwanted barrier hampering the uptake of for instance nutrition and/or medicines by a patient. This is for instance a major problem for patients suffering from cystic fibrosis, COPD, asthma, bronchitis, and tuberculosis. Uptake of nutrients in the gastrointestinal tract is insufficient because of the decreased permeability of the mucus layer covering the epithelial cells. This often results in failure to thrive. Likewise, pharmaceutical compositions are less able to reach their target, for instance in the lungs, because of a thick mucus layer. This reduces the efficiency of current treatment. Therefore there was a need for a means of decreasing the mucus barrier in a mammal.

### SUMMARY OF THE INVENTION

The present invention provides a recombinant or substantially isolated or purified mammalian mucinase, or a modified form thereof having a substantially similar mucin-hydrolyzing activity. The invention also provides a recombinant or substantially isolated or purified mucinase, said mucinase being a mucinase having an amino acid sequence essentially corresponding to the amino acid sequence shown in FIG. 8, or a modified form of said mucinase having a substantially similar mucin-hydrolyzing activity. A mucinase of the invention is particularly suitable for degrading mucus. Said mucinase is particularly suitable of degrading mucus in a mammal, because a mammal naturally comprises a mucinase of the invention, which is endogeneously present in said mammal. Therefore, a mucinase of the invention does not provoke severe side-effects and harmful immune responses in said mammal.

With a mucinase of the invention, it is now for instance possible to efficiently decrease an unwanted mucus barrier in the respiratory tract and/or gastrointestinal tract of a patient suffering from a disease in which mucus is involved without harmful side-effects. On the one hand, degrading mucus improves the capability of said patient to take up oxygen and/or nutrients. On the other hand, by (partially) degrading a thick mucus layer, delivery of other pharmaceutical compositions becomes easier, and mucociliary clearance improves, resulting in fewer persistent infections. It has also become possible to selectively counteract tumor cells which have a different mucus expression pattern as compared to normal cells. Additionally, it is possible to at least in part degrade micro-organisms comprising mucus.

The invention also provides a mucinase of the invention, produced by a host or host cell and isolated from said host or host cell or medium in which said host cell is cultured. In one embodiment, the amino acid sequence of said mucinase is encoded by a nucleotide sequence essentially corresponding to the nucleotide sequence shown in FIG. 8. Said mucinase is called AMCase. Preferably, said mucinase has a molecular weight of about 50 kDa.

By a mucinase is meant herein a proteinaceous molecule which is capable of, at least in part, hydrolyzing a mucin. This results in the cleavage of at least one sugar moiety bond of said mucin. Preferably a sugar moiety, such as a β.1-4 linked N-acetylglucosamine is cleaved. In terms of the invention, by “substantially isolated or purified” is meant that said mucinase is removed from an environment in which it naturally occurs, or that a sample, comprising said mucinase, is enriched for said mucinase. Said sample may be obtained from a mammal, for instance from a mouse or a human individual, because mammals endogeneously comprise a mucinase of the invention.

By a recombinant mucinase is meant a mucinase which has artificially been made, as opposed to mucinases which are naturally generated in living organisms. A recombinant mucinase can for instance be generated by expression, either in vitro or in vivo, of a vector comprising a nucleic acid sequence encoding said mucinase.

In terms of the invention, “a sequence essentially corresponding to” means that variations of said sequence are allowed, as long as said variations do not alter the properties of said sequence in kind. Said properties may however be somewhat altered in amount. For amino acid sequences, said variations for instance include a conservative substitution: a substitution of an amino acid residue with another amino acid residue with generally similar properties (such as size and hydrophobicity) such that the functioning of said amino acid sequence remains the same in kind, not necessarily in amount. Additionally, an amino acid residue may be deleted without significantly altering the function of the amino acid sequence. Generally, said sequence variations will be limited to less than 35%, preferably less than 20%, more preferably less than 10%. Therefore, the variants will generally have a homology of 65%, preferably 80%, more preferably 90%. An amino acid sequence essentially corresponding to a mucinase for instance has the same kind of mucin-hydrolyzing property as said mucinase, though not necessarily in amount. Likewise, a nucleotide sequence essentially corresponding to a nucleotide sequence shown in FIG. 8 has the same kind of properties of said nucleotide sequence shown in FIG. 8. It encodes for instance a mucinase. By “a modified form of said mucinase having a substantially similar mucine-hydrolyzing activity” is meant a molecule having substantially similar mucine-hydrolyzing activity, although said modified form may differ significantly from said mucinase. Said modified form may for instance comprise a functional part of said mucinase. In terms of the invention, a functional part of a mucinase is defined as a part which has a substantially similar mucin-hydrolyzing activity as said mucinase. Said functional part could for instance consist of the catalytic domain of said mucinase. Said modified form may also be a functional derivative, wherein several domains are deleted and/or substituted and/or added. For instance, said modified form may comprise a fusion protein. Said fusion protein, which is also herewith provided, preferably comprises a mucinase of the invention and/or a functional part thereof, and a protection moiety. Said protection moiety allows for a longer half-life as compared to an unprotected mucinase of the invention and/or an unprotected functional part thereof. Said protection moiety for instance comprises at least part of an immunoglobulin chain, preferably a constant region of said chain. In one embodiment of the invention, a fusion protein of the invention comprises a human mucinase of the invention and/or a functional part thereof. A fusion protein of the invention preferably retains mucinase biological activity, both in vitro and in vivo, and preferably has an improved pharmakinetics when administered in vivo as compared to an unprotected mucinase of the invention and/or an unprotected functional part thereof. Said fusion protein can also comprise several copies of a desirable domain of said mucinase, and/or an additional domain which is not derived from said mucinase.

By a substantially similar mucin-hydrolyzing activity is meant herein the same mucin-hydrolyzing activity in kind, not necessarily in amount. Like mucinase, a compound with a substantially similar mucin-hydrolyzing activity is capable of cleaving at least one sugar moiety bond of mucin. Said substantially similar mucin-hydrolyzing activity does not necessarily comprise additional (enzymatic) activities against other kind of compounds. If for instance a mucinase of the invention comprises other catalytic activities besides its mucin-cleavage activity, a compound comprising a substantially similar mucin-hydrolyzing activity does not necessarily comprise said other catalytic activities.

In one aspect the invention provides a pharmaceutical composition comprising an effective amount of a mucinase of the invention and a pharmaceutically acceptable carrier or diluent. Said pharmaceutical composition is particularly suitable for therapeutic or prophylactic treatment of an individual against a disease in which mucus is involved, such as for instance cystic fibrosis, COPD, asthma, bronchitis, tuberculosis, a mucin-producing tumour and/or infection by a protozoan parasite. Preferably, said pharmaceutical composition further comprises a therapeutically or prophylactically effective amount of a second pharmaceutical composition, such as human DNAse1, a mucolytic (eg n-acetylcysteine), an antibiotic (eg Tobramycin), a pancreatic enzyme supplement, an antifungal drug (eg itraconazole, caspofungin), an antihistamine, a bronchodilator, a leukotrien inhibitor, and/or a corticosteroid.

In terms of the invention, a disease in which mucus is involved means that said disease is either associated with an altered mucin expression pattern in a patient, and/or associated with a micro organism comprising mucus such as a protozoan parasite. Said altered mucin expression pattern may lead to a thick mucus layer in the respiratory tract and/or gastrointestinal tract hampering the uptake of oxygen and/or nutrients, and facilitating infections Alternatively, said altered mucin expression pattern may only be induced locally. This is for instance the case with carcinoma cells having an altered expression pattern as compared to normal cells.

The invention further comprises a composition comprising a mucinase of the invention and a carrier or diluent. For instance, said composition can be a medium for culturing cells, or a cosmetic, dental or food product.

Furthermore, the invention provides a method of therapeutic or prophylactic treatment of an individual against a disease in which mucus is involved, such as cystic fibrosis, COPD, asthma, bronchitis, tuberculosis, a mucin-producing tumour and/or infection by a protozoan parasite, comprising administering to said individual a pharmaceutical composition of the invention. The mucinase present in a pharmaceutical composition of the invention is capable of cleaving mucins. Therefore, with a method of the invention it has for instance become possible to decrease an unwanted mucus barrier in the respiratory tract and/or gastrointestinal tract of a patient suffering or at risk of suffering from a disease in which mucus is involved. It has also become possible to specifically bind and/or cleave mucus of tumor cells having an altered mucus expression pattern. Once bound, said tumor cell can be subject to additional treatment by conventional pharmaceuticals. As another example, it has now also become possible to protect and/or treat an individual against a mucus-comprising pathogen, by prophylaxis and/or treatment according to a method of the invention

The invention also provides a process for preparing a mucinase of the invention, or a modified form thereof having a substantially similar mucin-hydrolyzing activity, comprising growing a host or a host cell capable of producing said mucinase or modified form thereof and isolating the mucinase produced from said host or host cell or from medium in which said host cell is cultured. In one aspect of the invention, said host or host cell is genetically engineered. Preferably, the amino acid sequence of said mucinase is encoded by a nucleotide sequence essentially corresponding to the nucleotide sequence shown in FIG. 8.

In another aspect of the invention, a mucinase of the invention is provided which further comprises a chitin-hydrolyzing activity. By a chitin-hydrolyzing activity is meant herein a capability of cleaving at least one bond of chitin. Said mucinase is very suitable for degrading chitin, for instance chitin from pathogenic micro-organisms. Therefore the invention also provides a pharmaceutical composition for therapeutic or prophylactic treatment of an individual against infection by a chitin-containing pathogen, comprising a therapeutically or prophylactically effective amount of a mucinase of the invention and a pharmaceutically acceptable carrier or diluent. The invention also provides a method of therapeutic or prophylactic treatment of an individual against infection by a chitin-containing pathogen, comprising administering to said individual said pharmaceutical composition. Said method is for instance very suitable for treating a CF-patient comprising Aspergillus species in its respiratory tract. With a method of the invention, a thick mucus layer and Aspergillus species can be degraded in the respiratory tract simultaneously, because of both mucus-hydrolyzing as well as chitin-hydrolyzing activity of said pharmaceutical composition. Said method is also suitable for treatment of other pathogens in a mucosal lining, like for instance vulvovaginitis, and ringworm,

A composition comprising a mucinase of the invention and a carrier or diluent is also herewith provided. For instance, said composition may be a medium for culturing cells, in particular human cells, or a cosmetic, dental, or food product. Furthermore, the invention provides a chitin-based article of manufacture comprising a chitin-hydrolyzing amount of a mucinase of the invention. Said chitin-based article of manufacture may be a drug-containing drug carrier, an implant for controlled drug release or a transient functional implant.

An isolated host cell capable of producing a mammalian mucinase of the invention is also herewith provided, as well as a recombinant nucleic acid comprising a nucleotide sequence encoding, or complementary to a nucleotide sequence encoding, an expressable mammalian mucinase of the invention. Said mucinase may comprise an amino acid sequence essentially corresponding to the amino acid sequence shown in FIG. 8. Preferably, said nucleotide sequence essentially corresponds to, or essentially is complementary to, the nucleic acid sequence shown in FIG. 8. By “a nucleotide sequence encoding an expressable mucinase” is meant herein a nucleotide sequence encoding a mucinase that at least in part can be obtained by transcription and/or translation of said nucleotide sequence. By “essentially complementary to a nucleic acid sequence” is meant that a particular nucleic acid can bind by hybridisation to said nucleic acid sequence, especially under stringent conditions. By “essentially corresponds to the nucleic acid sequence shown in FIG. 8” is meant herein that a nucleotide sequence codes for the same amino acid sequence that is encoded by the nucleic acid sequence shown in FIG. 8, and/or codes for a modified form of said amino acid sequence having a substantially similar mucin-hydrolyzing activity. A nucleotide sequence coding for said same amino acid sequence can for instance utilize a different codon usage.

Also provided herewith is an oligonucleotide of at least about 8 nucleotides having a nucleotide sequence corresponding to, or complementary to, a nucleotide sequence shown in FIG. 8 and being capable of binding by hybridisation under stringent hybridisation conditions to nucleic acid coding for a mucinase of the invention. Said oligonucleotide is useful for different purposes. For instance, said oligonucleotide can be used as a probe in a hybridisation analysis, or as a primer in a nucleic acid amplification method such as PCR, NASBA, etc. The invention also provides a peptide of at least about 8 amino acid residues having an amino acid sequence derived from the amino acid sequence shown in FIG. 8 and representing or mimicking an epitope of a mucinase of the invention, in particular those having an amino acid sequence corresponding to an amino acid sequence shown in FIG. 8 and having antigenicity. Usually, such peptides will have a length of at least about 10, or even at least about 15, or at least about 40 amino acid residue. Preferably, said peptide comprises a length of about 30 amino acid residues. Said peptides are for instance suitable for diagnostic purposes, or in immunization protocols to raise mammalian mucinase-specific antibodies.

An antibody capable of binding to a mucinase of the invention is also herewith provided. Preferably, said antibody is a monoclonal antibody. An antibody of the invention can be used for many purposes, for instance for isolating and/or purifying (e.g. by affinity chromatography) a mucinase of the invention.

In yet another aspect the invention provides a diagnostic kit comprising an antibody of the invention, and/or a peptide of the invention, and/or a diagnostically effective amount of a mucinase of the invention, and a conventional component of diagnostic kits for detecting an antigen or an antibody. Also provided is a diagnostic kit comprising an oligonucleotide of the invention and/or a recombinant nucleic acid of the invention, and a conventional component of diagnostic kits for detecting a nucleic acid.

Furthermore, the invention provides a method of decomposing mucin comprising contacting said mucin with a mucinase of the invention under mucin-hydrolyzing conditions. The invention also provides a method of decomposing chitin comprising contacting said chitin with a mucinase of the invention which further comprises a chitin-hydrolyzing activity, under chitin-hydrolyzing conditions.

### LEGENDS OF THE DRAWINGS

FIG. 1. Isoelectric focusing profile of chitinolytic activity in mouse lung extract. Isoelectric focusing was performed as described in experimental procedures. Chitinolytic activity was measured using 4MU-chitotrioside substrate. The enzyme activity present in the different isoelectric focusing fractions is expressed as percentage of the total activity present in all fractions.

FIG. 2. Mouse AMCase cDNA sequence and deduced amino acid sequence. The cDNA sequence (GenBank Accession Number AF290003) is indicated by the upper sequence and the deduced amino acid sequence is depicted below the nucleotide sequence. The characteristic hydrophobic signal peptide (amino acids 1-21) is underlined with a single line. The putative chitin binding domain (amino acids 426-473) is underlined with a double line. The hinge region separating the catalytic domain from the chitin binding domain is underlined with a dashed line. The part of the protein purified from mouse intestine that was determined by Edman sequencing is boxed.

FIG. 3. Degradation products with colloidal chitin as substrate. The FACE technique (described in experimental procedures) was used to visualize the cleavage products of recombinant human chitotriosidase and recombinant mouse AMCase using colloidal chitin as substrate. Lane 1, no enzyme added. Lane 2, products formed after incubation with 50 kDa recombinant human chitotriosidase with chitin. Lane S, products formed with recombinant mouse AMCase and chitin. Lane 4, human chitotriosidase incubated without substrate. Lane 5, mouse AMCase incubated without substrate. Marker lane is indicated with M (sugar polymers are indicated on the right-hand side).

FIG. 4. Electrophoretic behavior of chitinases.

Panel A: Purified recombinant human chitotriosidase and mouse AMCase were separated on a 12.5% SDS-PAGE gel in the presence or absence of a reducing agent, and visualized by silver staining as described in experimental procedures (panel A). Lane 1, recombinant mouse AMCase under reducing conditions. Lane 2, recombinant human chitotriosidase under reducing conditions. Lane 3, recombinant human chitotriosidase under non-reducing conditions. Lane 4, recombinant mouse AMCase under non-reducing conditions. M indicates the molecular weight standards (mass (kDa) indicated at the left-hand side).

Panel B: The same purified recombinant enzymes as described in panel A were separated on a 10% SDS-PAGE gel containing glycol-chitin as described in experimental procedures. Chitinolytic activity was visualized as clearing zones in the gel. Lane 1, recombinant human 39 kDa chitotriosidase. Lane 2, recombinant human 50 kDa chitotriosidase. Lane 3, recombinant mouse AMCase (mass (kDa) indicated at the right-hand side).

FIG. 5. Effects of acidic pH.

Panel A: pH activity profile of the different chitinases. The pH optima were determined by monitoring enzyme activity at the indicated pH in McIlvaine buffer. Purified human recombinant chitotriosidase (closed lozenge), purified mouse AMCase (closed circle).

Panel B: Effects of acidic pre-incubation. Purified recombinant human chitotriosidase and mouse AMCase were pre-incubated for 30 minutes at the indicated pH in McIlvaine buffer prior to enzyme activity measurement at the assay pH (see experimental procedures). Activity prior to incubation at the indicated pH is defined as 100%.

Panel C: Precipitation by trichloroacetic acid (TCA). Purified recombinant human chitotriosidase and mouse AMCase were incubated with the indicated percentages of TCA. The amount of remaining enzyme activity after centrifugation is shown as percentage of initial amounts.

FIG. 6. Tissue distribution of mouse AMCase mRNA.

Panel A: The relative expression levels of mouse AMCase in various mouse tissues as determined by dot blot analysis using a RNA Master Blot (Clontech) as described in experimental procedures. The highest level of expression is defined as 100%.

Panel B: Northern blot of RNA isolated from the indicated mouse tissues. 15 microgram total RNA was separated on an agarose gel as described in experimental procedures. The full length mouse AMCase cDNA was used as probe. As a control for RNA loading a glyceraldehyde-3-phophate dehydrogenase (GAPDH) probe was used (data not shown). The position of the 18S ribosomal RNA band is indicated.

FIG. 7. Tissue distribution of human AMCase mRNA. The relative expression levels of human AMCase in various human tissues was determined by dot blot analysis using a RNA Master Blot (Clontech) using the oq35c04,s1 EST clone (GenBank Accession Number AA976830) as probe. The highest level of expression is defined as 100%. Several tissues were excluded from the figure since they did not result in detectable signal: amygdala, caudate nucleus, cerebellum, cerebral cortex, frontal lobe, hippocampus, medulla oblongata, occipital lobe, putamen, substantia nigra, temporal lobe, thalamus, nucleus accumbeus, spinal cord, fetal brain, fetal heart, fetal kidney, fetal liver, fetal spleen and fetal thymus.

FIG. 8. Human AMCase cDNA sequence and deduced amino acid sequence.

Panel A: The human AMCase cDNA sequence (GenBank Accession Number AP290004) is indicated by the upper sequence and the deduced amino acid sequence is indicated below the nucleotide sequence. The characteristic hydrophobic signal peptide (amino acids 1-21) is underlined with a single line.

Panel B: Amino acid sequence comparison of mature (without signal peptide) human (h) and mouse (m) AMCase and human chitotriosidase. Residues conserved among at least two out of the three sequences are boxed.

FIG. 9. The effect of AMCase on glycoproteins was studied by evaluating the effect of mouse AMCase on mucin, a type of glycoproteins expressed on mucosal surfaces in the airways and gastrointestinal tract. Bovine submaxillary gland mucin (Sigma) 125 μg was dissolved in 40 μl 0.05M NaAc pH 5.0 in the presence or absence of 500 ng mouse AMCase. After overnight incubation at 37′, the mucin was electrophorized on an SDS-PAGE gel 7.5%, followed by silver staining. As is shown in FIG. 9, the mucin was degraded substantially by treatment with the AMCase (left lane), but not when it was treated with control buffer (right lane).

### DETAILED DESCRIPTION OF THE INVENTION

Mucus provides an important defensive barrier which forms the first line of defence to the external environment. However, several diseases involve a disturbed generation of mucus, resulting in thick and adhesive mucus. This leads among others to problems according to the uptake of oxygen and nutrients by a patient. Additionally, because of said thick adhesive mucus, current pharmaceutical compounds have a reduced capability to reach their targets (for instance epithelial cells or DNA present in said mucus), and opportunistic infections occur as a result of impaired mucociliary clearance.

Furthermore, infections with mucus-containing pathogens like protozoan parasites can induce severe complications. The same applies to several other pathogens present in mucosal linings of an individual, for instance the fungus Aspergillus, in the lungs.

Although pharmaceutical compositions are currently used to counteract diseases in which mucus is involved, there is yet no efficient and satisfactory method to degrade unwanted mucus.

For cystic fibrosis and COPD, the most widely used mucolytic agents are nacetylcysteine or acetylcysteine, and recombinant human DNAse. Other mucolytics used in clinic include guaifenesin, carbocisteine lysine, citiolone, sobrerol, ambroxol, myrtol, iodinated glycerol, isobutyrylcysteine, and letosteine.

N-acetylcysteine is an aerosolized mucolytic agent often used as adjunctive therapy for pulmonary complications of cystic fibrosis. The viscosity of mucous secretions in the lungs is dependent upon the concentrations of mucin and DNA. N-acetylcysteine acts to splits the sulfide bonds between DNA and mucins thereby decreasing mucus viscosity. The action of N-acetylcysteine is pH dependent. Mucolytic action is significant at ranges of pH 7-9 (Kastrup et al 1998).

Adverse effects reported with acetylcysteine include stomatitis, nausea, vomiting, hemoptysis, and severe rhinorrhea. Acetylcysteine has an unpleasant, pungent odor that may lead to an increased incidence of nausea. Bronchoconstriction has also been reported with acetylcysteine therapy. N-acetylcystein has mucolytic activity in vitro, this activity has however not been demonstrated convincingly in vivo (Celli et al 1995). A possible explanation for this finding is a pH in the airways that is lower than 7.0. Even though the normal tracheal mucus pH ranges from 6.9-9.0, in infection pH values can become as low as pH 5.8 in mucus (Buhrmester 1933). An acidic pulmonary environment has been reported for at least two mucus-associated lung diseases, namely asthma (Hunt et al.) and cystic fibrosis (Choi et al). A similar decreased pH may also be present in the lungs of COPD patients, as they have respiratory ascidosis (Plant et al.).

DNA is a factor that contributes to viscous mucus in CF patients. This high extracellular DNA (as high as 3-14 mg/ml, Chernick and Barbero 1959, Potter et al 1960) content further thickens airway secretions. Recombinant human DNase (Pulmozyme) has been demonstrated to reduce the viscosity of sputum in CF patients by hydrolyzing the extracellular DNA (Shak et al 1990, Zahm et al 1995). DNase is a highly purified solution of recombinant human deoxyribonuclease I (rhDNase), an enzyme that selectively cleaves DNA. Studies have demonstrated that daily administration of recombinant human DNase resulted in definite improvement in pulmonary function, as assessed by FEV1, above baseline (Fuchs et al 1994, Shak et al 1995). Recombinant human DNAse is indicated in the management of patients with CF to improve pulmonary function and decrease the frequency of respiratory infections. Safety and efficacy have not been demonstrated in children less than 5 years of age. The recommended dose of recombinant human DNase for most patients with CF is 2.5 mg by nebulization once daily. Adverse effects include voice alteration, pharyngitis, laryngitis, rash, and chest pain. As DNAse only targets the DNA present in viscous sputum, the mode of action of AMCase is independent of DNAse, and possibly synergistic. In addition, for indications as COPD, where less DNA is present in the sputum, AMCase is more effective.

Although pharmaceutical compositions are currently used to counteract diseases in which mucus is involved, it has to be concluded that there is an unmet need for more sophisticated agents to degrade thick, adhesive mucus and to treat and/or prevent chronic infections with pathogens in epidermal and mucosal body linings. Preferably such agents should be highly specific and effective and should not be prone for developing resistance against and neither cause toxic side effects.

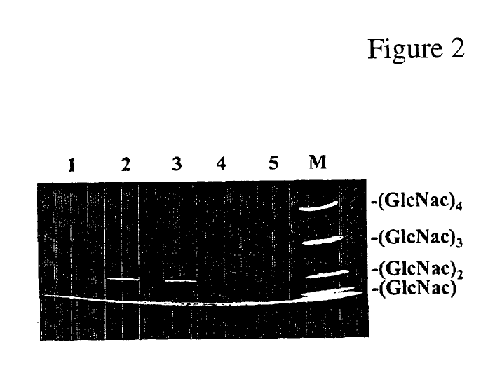

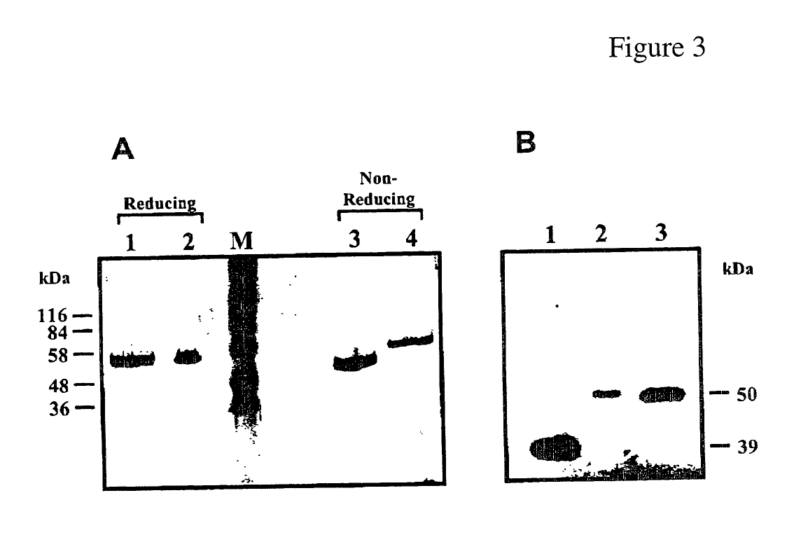

It was previously disclosed that bacterial chitinase preparations, which are expected to be highly immunogenic when administered to humans, can degrade human ocular mucus (Argueso et al 1998). Here it is disclosed that mammals naturally comprise a mucinase. Said mammalian mucinase, endogeneously present at mucosal surfaces in mammals, provides the solution for the above-mentioned problems concerning diseases in which mucus is involved. A mucinase of the invention is capable of cleaving mucus. In one embodiment a specific mouse and human mucinase is provided, called AMCase, which is depicted in more detail in FIGS. 2 and 8. It has a catalytically active 39 kDa domain which is connected via a hinge region with a C-terminal mucin binding domain. To show its mucus-degrading property we have incubated a mixture of submaximallary gland mucins with recombinant 50 kDa AMCase. The effect of the incubation was examined by analysis with SDS-PAGE and silver staining. FIG. 9 shows that a remarkable reduction in size of the mucins occurs. It was noted that the viscosity of the mucin solution was markedly reduced following incubation with AMCase. The flow rate of the mucin solution, as measured in a vertically positioned glass pipette, was increased with almost a factor of 2.

A thick and adhesive mucus layer in the respiratory tract and/or gastrointestinal tract of an individual with for instance CF, COPD, or asthma can be, at least in part, degraded by administration of a mucinase of the invention to the respiratory tract and/or gastrointestinal tract of an individual. (Partly) degradation of said thick mucus layer enhances oxygen and/or nutrient uptake by said individual. Additionally, it enhances the capability of other medicaments to reach their target, and prevents the occurence of persistent chronic infections. Because a mucinase of the invention is endogeneously present in mammals, it does not provoke severe immunological reactions after additional administration of said mucinase to said mammals.

A pharmaceutical composition or commercial formulation typically comprises a certain percentage of Active Pharmaceutical Ingredient, i.e. purest achievable form of a mucinase of the invention, as well as a suitable carrier that ensures the composition to be an appropriate delivery vehicle to the area of disease. Examples are a cream or ointment for skin infections, tablet or capsule formulations for the digestive tract, and an inhalation formulation for the pulmonary airways. Appropriate doses will be formulated such that these are the Maximum Effective Dose with the appropriate safety and tolerability profile.

In order to use a mammalian mucinase, like AMCase, as a pharmaceutical agent against thick mucus layers, the mammal has to be tolerant for said mucinase. Because a mucinase of the invention occurs naturally in the mammalian body, no strong immune response is elicited by additional administration of the enzyme to said mammal.

Another requirement in the application of an enzyme as therapeutic agent is its ability to survive and to be functional in the body. This requirement is met because a mucinase of the invention is a remarkably stable enzyme. For instance, mouse AMCase can endure incubation at acidic (pH 2) upto quite basic conditions (pH 8). The enzyme is quite resistant against various proteases.

A mucinase of the invention preferably has a low pH optimum. For instance, mouse AMCase shows a pronounced pH optimum at pH 2.3 and a less pronounced optimum at pH 4-7. A mucinase of the invention with a low pH optimum is very suitable, especially because it is pH stable, for enhancing food digestion and normal bowel movement in patients with a thick mucus layer in the gastrointestinal tract, like or instance cystic fibrosis patients. It can for instance well perform its catalytic action in the acidic stomach, whereas many other pharmaceutical compounds are inactivated.

A mucinase of the invention can be combined with existing medicaments. Once (part of) a thick mucus layer is cleaved and/or bound by mucinase, other pharmaceuticals are more capable of reaching their targets. In one embodiment the invention therefore provides a mucinase conjugate comprising a mucinase of the invention and a second molecule, like for instance a second pharmaceutical compound and/or an immunoglobulin chain. Said mucinase is preferably bound to said second molecule. Said conjugate is suitable for a combined therapy. For instance, said conjugate can cleave mucus in the lungs and/or gastrointestinal tract of a patient with its mucin-hydrolyzing catalytic domain, after which said second pharmaceutical compound is capable of performing its therapeutic task. Alternatively, said conjugate can bind mucus with its mucus-binding domain without cleaving said mucus, after which said second pharmaceutical compound is capable of performing its therapeutic task.

Possible AMCase combination therapies are:

CF: DNAse 1 (pulmozyme, Genentech) and AMCase

Oral AMCase and pancreatic enzyme supplemements

AMCase and antibiotics

AMCase and gene therapy

CF with ABPA: itraconazole (or other antifungal treatments, for ABPA itraconazole is most often used) with AMCase (+other CF medication)+/− oral corticosteroids

Asthma: AMCase with antihistamines, bronchodilators or corticosteroids.

Asthma with ABPA: itraconazole (or other antifungal treatments, for ABPA itraconazole is most often used) with AMCase+/−antihistamines, bronchodilators or corticosteroids

ABPA without asthma or CF: oral corticosteroids and AMCase (topical administration)

Chronic obstructive pulmonary disease with bronchitis: AMCase alone, or in combination with bronchodilators, b-adrenergic agents, methylxanthines, corticosteroids, or mucolytics, (mostly with n-acetylcysteine: fluimocyl or mucomyst) is efficacious.

Systemic indications for a mucinase of the invention are also foreseen. These include but are not limited to mucus producing benign and malignant tumours. Mucinase can be administered alone or in combination with other treatments.

A mammalian mucinase can additionally comprise chitin-hydrolyzing activity. For instance, we have found that mouse and human AMCase also comprise chitin hydrolyzing activity. This is an important finding, because 6% of the human individuals is deficient for the only human chitin-hydrolyzing enzyme known, chitotriosidase. In those individuals, an endogenic mucinase can take over that function.

Next to cellulose, chitin is the most abundant glycopolymer on earth, being present as a structural component in coatings of many species, such as the cell wall of most fungi (Debono and Gordee 1994), the microfilarial sheath of parasitic nematodes (Fuhrman and Piessens 1985, Araujo et al 1993), the exoskeleton of all types of arthropods (Neville et al 1976) and in the lining of guts of many insects. (Shahabuddin and Kaslow 1994). Chitinases (EC 3.2.1.14) are endo-β-1,4-N-acetylglucosaminidases that can fragment chitin and have been identified in several organisms (Flach et al 1992). Until a few years ago it was generally assumed that man lacks the ability to produce a functional chitinase.

Chitin-containing pathogens like fungi constitute a common treat of infection of mammalians. The mammalian immune system governs a broad array of defence mechanisms against systemic fungal infections. However, the incidence of life-threatening systemic fungal infections is rapidly increasing as the result of increasing active suppression of the immune system of patients during medical interventions, for example during chemotherapies and transplantations, as well as due to viral suppression of the immune system, for example during AIDS. Present antifungal agents show serious limitations such as the increasing resistance among the major human pathogenic fungi against existing drugs like azoles or because of the limited efficacy and toxic side effects of antifungal compounds like amphotericins. It has therefore to be concluded that there is an unmet need for more sophisticated agents to treat and/or prevent chronic infections with chitin-containing pathogens in epidermal and mucosal body linings. Preferentially such agents should be highly specific and effective and should not be prone for developing resistance against and neither cause toxic side effects.

The features of a mucinase of the invention which further comprises chitin-hydrolyzing activity (endogenous production in mucosal body linings, extreme acid pH stability and protease resistance, potent fungistatic action) make said mucinase an ideal candidate for use as drug against topical/mucosal infections with chitin-containing pathogens such as fungi.

As chitin-containing pathogens such as fungi enter the body via accessible sites such as nose, mouth, lungs, ears, eyes, skin, urethra, bladder, topical infections of these sites occur frequently. These sites are in contact with the outside world, and as a result, topical administration of antifungal drugs may be necessary to achieve optimal efficacy.

Prominent topical fungal infections for AMCase indication are for instance allergic bronchopulmonal aspergillosis (ABPA), vaginal infection with candida (vulvovaginitis: Low pH; a mucinase of the invention is active whereas chitotriosidase is not), dermatophytosis (ringworm of skin, scalp, nails and athlete's foot), and other topical fungal infections in body linings (including non allergic lung aspergillosis).

Current treatment of fungal infections include allylamines (mostly topical applications) antimetabolites (oral, IV), azoles (topical, oral, IV), glucan synthesis inhibitors (IV), polyene macrolides (mostly IV, amphotericin B and derivatives, nystatin topical, pimaricin ophtalmic), and other drugs (systemic: Griseofulvin, topical: ciclopiroxolamine, haloprogin, tolnaftate, undecylenate). Many of these drugs, especially the ones for invasive and systemic infections are hampered by serious side effects. Such side effects are less likely if a mucinase of the invention which further comprises chitin-hydrolyzing activity, which is endogenously expressed, is used as a protein drug. Thus, a mucinase of the invention which further comprises chitin-hydrolyzing activity is very suitable for treatment of lung infections in for instance cystic fibrosis and COPD.

Allergic bronchopulmonary aspergillosis (ABPA) is a syndrome seen in patients with severe obstructive lung disease, most commonly in asthma and cystic fibrosis (Cockrill & Hales 1999). Chronic colonization of the airways by Aspergillus is apparent in these patients, accompanied by Aspergillus-specific IgE antibody production and eosinophilia. ABPA is generally treated by using oral corticosteroids such as prednisone to suppress the inflammatory component of the disease. Antifungal therapy, such as treatment with the orally active Itraconazole, is has been reported. However, no controlled trials have been reported. In addition, as Itraconazole is used for systemic treatment, Aspergillus in the airway lumen may not be treated as efficiently. Therefore, novel treatments need to be developed to treat fungal infections in these pulmonary diseases. A mucinase of the invention which further comprises chitin-hydrolyzing activity is suitable for use for this indication.

A mucinase of the invention which further comprises chitin-hydrolyzing activity is also suitable in the event of resistance to current antifungal therapies. For instance, flucytosine resistence is common, especially when used as monotherapy for candida. Adverse effects are displayed as well. Resistance to ketoconazole described upon prolonged treatment of AIDS patients, adverse effects indications mucosal candidiasis, mycoses, histoplasmose, dermatophytes. Resistance to flucanazole is increasing in AIDS patients and adverse effects are displayed, indications are mucosal candidiasis, vulvovaginal candidiasis. Resistance to itraconazole is not clear, few side effects, indication candida histoplasmosis, mycoses, aspergillosis, sporotrichosis, candida Terbinafine, esp. to dermatophytes

A mucinase of the invention which further comprises chitin-hydrolyzing activity can also be administered in combination with another current pharmaceutical. The invention therefore also provides a mucinase conjugate, comprising a mucinase of the invention which further comprises chitin-hydrolyzing activity and a second molecule, for instance a second pharmaceutical compound and/or an immunoglobulin chain. Preferably, said mucinase is bound to said second molecule. Said mucinase conjugate can bind chitin with its chitin-binding domain after which said second pharmaceutical compound is capable of performing its therapeutic task. Said mucinase can also cleave said chitin with its chitin-hydrolyzing catalytic domain, but this is not necessary. A possible combination therapy for treatment of a fungus infection is: Azoles, antimetabolites, glucan synthesis inhibitors, griseofulvin (all with intracellular activity) together with a mucinase of the invention which further comprises chitin-hydrolyzing activity, like for instance AMCase. Of course, the several pharmaceutical compositions do not necessarily have to be administered at the same time. They can be administered together or separately, with either the same or different administration doses and administration intervals.

In one aspect, the invention provides a mucinase of the invention which has a low pH optimum. A major advantage of a mucinase of the invention which has a low pH optimum is that said mucinase even withstands the harsh conditions in the gastrointestinal tract and can therefore be administered orally.

A mucinase of the invention which has a low pH optimum is also suitable for use as topical agent. For instance, Athlete's foot, a topical infection caused by Trychophyton or Epydermophyton involves a local decrease of pH. Therefore, a mucinase of the invention which has a low pH optimum and which has a chitin-hydrolyzing activity is particularly suitable for topical treatment of Athlete's foot, and for topical treatment of any pathogen involving a low pH, like for instance a vaginal infection by Candida albicans. For instance, incubation of hyphae of Candida albicans with recombinant AMCase results in selective lysis of the growing tip, similar to the effect of chitotriosidase.

Additional important advantages of a mucinase of the invention which further comprises chitin-hydrolyzing activity compared to classic antifungal agents are the following. In the first place, since a mucinase of the invention which further comprises chitin-hydrolyzing activity is an endogenous protein, its administration will not result in a severe immune reaction. In the second place, resistance of fungi against chitinases has not been developed so far and seems intrinsically difficult given the fact that despite the evolutionary pressure plant fungal pathogens have remained sensitive to chitinases.

To counteract chitin-containing pathogens, like fungi, a mucinase of the invention which further comprises chitin-hydrolyzing activity, like AMCase, can be administered locally as a crème, for instance on the skin. It may also be used for treatment of local ear-infections or vagina-infections, such as vulvovaginitis. Especially the latter also involves an acidic environment, in which a mucinase of the invention which further comprises chitin-hydrolyzing activity and a low pH optimum, like AMCase, is very well capable to perform its catalytic actions.

Body locations of a mucinase of the invention has led us to the conclusion that said mucinase can perform additional functions next to mucus degradation and defence against chitin-containing pathogens. For instance, the remarkable high concentration of AMCase in the gastrointestinal tract involves a role in food processing during the evolution of mammals.

A chitin-hydrolyzing capability of a mammalian mucinase, for instance AMCase, can also be exploited as a tool to degrade injected or implanted chitin-based structures for medical purposes. For instance, drugs can be incorporated in chitin-based capsules. The concomitant presence of well defined amounts of a mammalian mucinase which further comprises chitin-hydrolyzing activity, in the capsule ensures a controlled release of drugs. A slow but gradual release of drug is particularly envisioned when said drugs is trapped in a chitin matrix. The use of a said mucinase in such a system results in ultimate destruction of the chitin-based capsule and does not elicit an immunological response. The drugs used in such a system can vary from small compounds to protein and DNA fragments for the purpose of enzyme and gene therapy. Chitin (or analogues thereof) is already employed as a carrier for drugs.

Another application is the use of a mammalian mucinase which further comprises chitin-hydrolyzing activity for the swift degradation of implants that contain chitin as a structural component. This is useful in the case of implants that only temporary have to fulfil a function and can be conveniently degraded by administration of a mammalian mucinase which further comprises chitin-hydrolyzing activity, like AMCase.

A mucinase of the invention which further comprises chitin-hydrolyzing activity and a low pH optimum, like AMCase, is especially suitable for the above mentioned applications in case that an acid environment is involved, or additional mucus degradation is required.

A mammalian mucinase which further comprises chitin-hydrolyzing activity can also be used ex vivo for degradation of mucus containing and/or chitin containing micro organisms. For instance, as a preventive measure a mucinase of the invention can be added to culture medium of cells. Said cells may preferably be cultured in the absence of antibiotics. Examples are the ex vivo culture of cells for the purpose of gene therapy and the ex vivo culture of keratinocytes to be used in connection with wound healing.

A mucinase of the invention is as well suitable as an additive in tooth paste and body lotions in order to prevent infections with mucus containing and/or chitin containing micro organisms. Additionally, a mucinase of the invention can be used as a food preservative. For instance, it can inhibit growth of mucus-containing and/or chitin-containing pathogens in food.

The N-terminal amino acid sequence of purified AMCase was determined (Boot et al 2000) (see FIG. 2). The N-terminal amino acid sequence allowed the cloning of the corresponding full length mouse AMCase cDNA, (Boot et al 2000). Said full length cDNA predicts the synthesis of a 50 kDa (pI 4.85) protein with a characteristic signal peptide (see FIG. 2). Expression of this cDNA in COS cells led to secretion of a 50 kDa active mucinase/chitinase with a pI of 4.8. AMCase was found to bind to chitin particles with high affinity. Chitin affinity chromatography was used to purify the enzyme, as described in experimental procedures. The procedure resulted in a 30.082-fold purification of an apparently homogeneous 50 kDa protein. The specific activity of the purified enzyme was 3.9 nmol 4-methylumbelliferyl. chitotrioside hydrolyzed at pH 5.2 per mg per hour, being almost identical to that of chitotriosidase. The catalytic domain of mouse AMCase is also herewith provided.

Mouse AMCase mRNA is predominantly found in stomach, submaxillary gland and also, at a lower level in the lung (see FIG. 6). Surprisingly, no mouse AMCase mRNA can be detected in the small intestine, suggesting that the protein in the intestine is probably derived from the upper parts of the gastrointesinal tract, such as the stomach.

Our findings demonstrate that AMCase in mammalians is distinct from chitotriosidase: the newly discovered, discrete enzyme is referred to as acidic mammalian chitinase or AMCase. AMCase is also present in man. Screening the human EST database at the NCBI with the acidic mouse chitinase cDNA, revealed the presence of a human EST clone (oq35c04.s1, Genbank acc. nr. AA976830) that is highly homologous to the acidic mouse chitinase. The tissue distribution of the human mRNA was examined using a human Masterblot (Clontech). The expression pattern of this mRNA is similar to the expression pattern of the acidic mouse chitinase (FIG. 6), being highly expressed in the stomach and at a lower level in the lung. Using degenerate oligonucleotides directed against members of the chitinase family, we were able to amplify other regions of the human acidic chitinase, generating enough information to clone the full-length human acidic chitinase cDNA. Screening the Genbank database using the full-length human cDNA revealed that it was almost identical to TSA1902-L and TSA1902-S from a lung cDNA library described by Saito et all (Saito et al 1999). These two sequences are most probably splice variants of the acidic human chitinase mRNA. Only expression of full length human AMCase cDNA in COS cells led to the production of a protein with chitinolytic activity. Sequence comparison of the human acidic chitinase and the mouse acidic chitinase revealed an 82% identity and a similarity of 86%. (compare FIGS. 2 and 8). The catalytic domain of human AMCase is also herewith provided.

Additional proof for the existence of two discrete genes encoding a phagocyte chitinase (chitotriosidase) and mucosal mucinase/chitinase (AMCase) is rendered by our finding that in man the former enzyme is encoded by a gene in locus 1q31 and the latter by a gene in locus 1p13.

A mucinase of the invention can be obtained by expression of a nucleic acid encoding said mucinase in a host or host cell, and subsequent isolation of said mucinase from said host or host cell or medium in which said host cell is cultured. Said host or host cell may be naturally expressing said mucinase. Alternatively, said host or host cell may be genetically engineered. A nucleic acid encoding said mucinase may be provided to said host or host cell.

A mucinase of the invention can also be obtained by substantially isolating or purifying said mucinase from an environment. Methods for isolating a proteinaceous molecule from an environment are known in the art (for instance chromatography) and need no further explanation here. A sample comprising said mucinase can for instance be enriched for said mucinase by applying said sample onto an affinity column and collecting an elution fraction enriched for said mucinase. Enrichment can also be performed by centrifugation and subsequent separation of a fraction enriched for said mucinase. A person skilled in the art is well capable of performing alternative isolation and/or purifications procedures, which are known in the art.

A nucleic acid encoding a mucinase of the invention, and/or a modified form thereof having a substantially similar mucin-hydrolyzing activity, is suitable for gene therapy. For instance, mammalian cells, preferably cells of a mucosal lining, can be provided with said nucleic acid. After that, said mucinase and/or modified form can be expressed, resulting in (increased) cleavage of mucus. In one embodiment, a cell which is transformed with said nucleic acid does not naturally produce a substantially amount of a mucinase of the invention. However, in another embodiment, said cell already produces said mucinase. In that case, production of mucinase can be enhanced by gene therapy with a nucleic acid of the invention. Gene therapy with a nucleic acid of the invention is for instance very suitable for inducing or enhancing mucinase expression in the lungs and/or gastrointestinal tract of a patient suffering from a disease in which mucus is involved.

Likewise, a nucleic acid encoding a mucinase of the invention which further comprises a chitin-hydrolyzing activity, and/or a modified form thereof having a substantially similar chitin-hydrolyzing activity, is suitable for gene therapy. For instance, mammalian cells, preferably cells of a mucosal lining, can be provided with said nucleic acid, resulting in (enhanced) expression of a mucinase of the invention which further comprises a chitin-hydrolyzing activity. Said mucinase is capable of counteracting chitin-containing pathogens present in said mucosal lining.

The invention will now be illustrated by the following examples which merely serve to exemplify the invention and are not intended to limit the scope of the invention.

### EXAMPLES

### Example 1

### Cloning and Composition of cDNAs Encoding Mouse and Human AMCase

Mouse AMCase

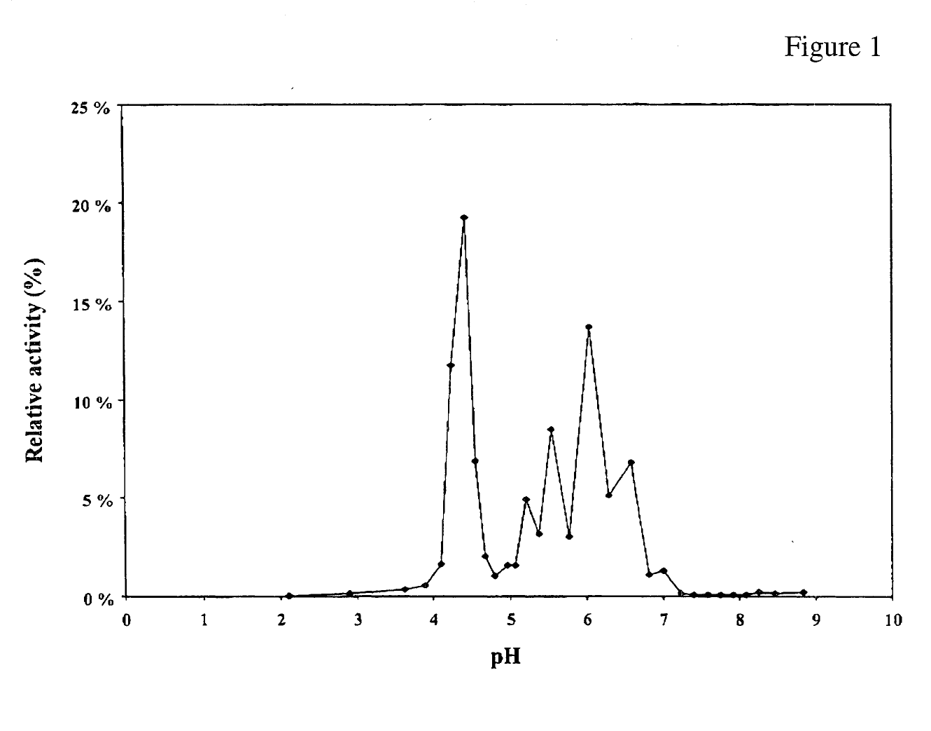

To obtain more insight into the potential occurrence of multiple mammalian chitinases, tissues of mouse and rat were examined for chitinolyic activity using the chitin-like 4-methylumbelliferyl-β-chito-oligosaccharide substrates. In extracts of stomach and intestine a high level of activity was detected, while extracts of lung, tongue, kidney and plasma showed significant but lower activities. Isoelectric focusing (by flatbed isoelectric focusing in granulated Ultrodex gels (Pharmacia) as described by Renkema et al 1995) of a mouse lung extract revealed a major peak of chitinolytic activity with pI 4.5 while minor peaks were found with pI's 5.5-6.5 (FIG. 1). Extracts of other mouse and rat tissues showed similar profiles of chitinolytic activity upon isoelectric focusing. The observed rodent chitinase with acidic isoelectric point (pI 4.5 form) differs strikingly from human chitotriosidase which has an apparent neutral/basic pI.

The mouse acidic chitinase activity was found to bind to chitin particles with high affinity. Chitin affinity chromatography was used to purify the enzyme. Detergent-free extracts of mouse tissues were prepared by homogenization in 10 volumes of potassium phosphate buffer pH 6.5, using an Ultra-turrax and centrifugation for 20 minutes at 15,000×g. The mouse intestine extract was adjusted to pH 5.0 by the addition of citric acid (0.2 M); NaCl was added to a final concentration of 2 M. A chitin column was prepared by mixing 10 grams swollen Sepharose G25 fine (Pharmacia, Uppsala, Sweden) with 300 mg of colloidal chitin prepared as described by Shimahara et al. (Shimahara et al 1988)., followed by equilibration with phosphate-buffered saline (PBS) containing 2M NaCl. The extracts were applied onto the column with a flow speed of 0.4 ml/minute. After extensive washing, bound chitinase was eluted from the column with 8M urea, that was subsequently removed by dialysis. Protein concentrations were determined according to the method of Lowry et al. Lowry et al 1951) using BSA as a standard. Fractions containing chitinase activity were subjected to SDS-PAGE and Western blotting as described (Renkema et al 1995).

The procedure resulted in a 30,000-fold purification of an apparently homogeneous 50 kDa protein. The specific activity of the purified enzyme was 3.9 nmol 4-methylumbelliferyl-chitotrioside hydrolyzed per mg per hour at pH 5.2, which is almost identical to that of human chitotriosidase. The N-terminal amino acid sequence of purified acidic chitinase was determined as described by (Renkema et al 1995) using a Procise 494 sequencer. (Applied Biosystems Perkin Elmer, Foster City, Calif., USA) (FIG. 2) and was found to be almost identical to that of other known members of the chitinase family. This amino acid sequence allowed the cloning of the corresponding full length mouse acidic chitinase cDNA, as described in experimental procedures. The full length cDNA predicts the synthesis of a 50 kDa (pI 4.85) protein with a characteristic signal peptide (FIG. 2). Transient expression of this cDNA in COS-1 cells was performed exactly as described previously (Boot et al 1995), and led to the secretion of an 50 kDa active chitinase with a pI of 4.8.

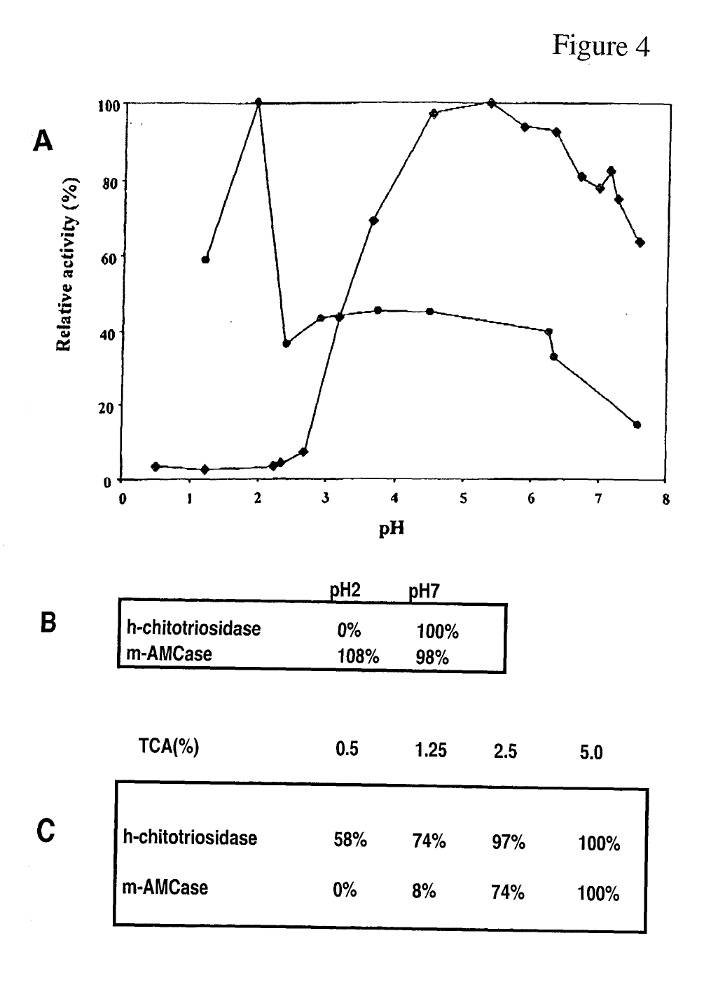

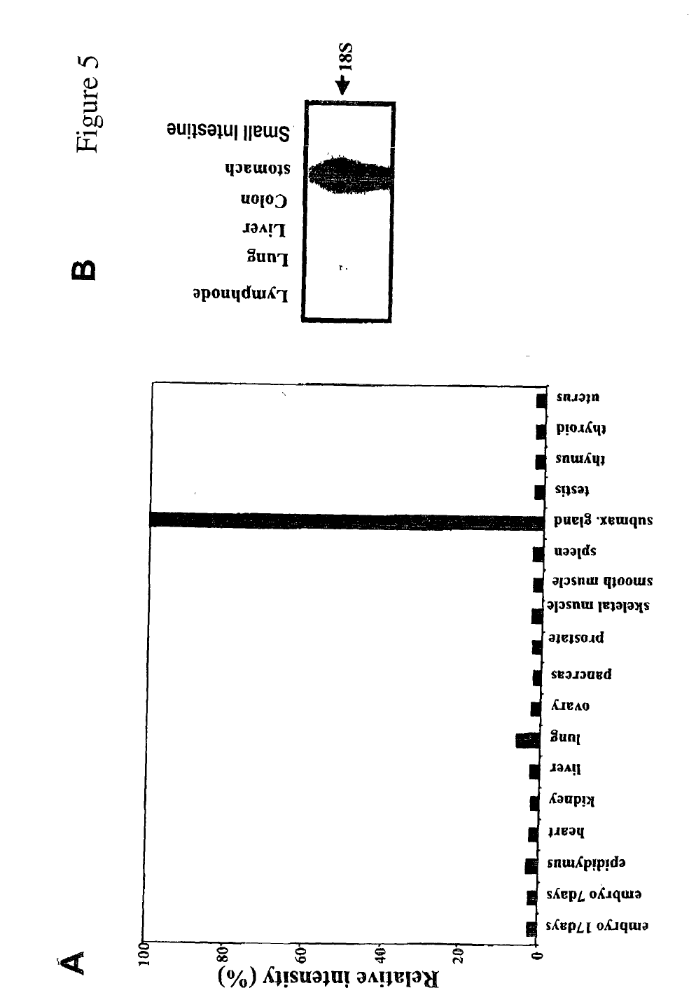

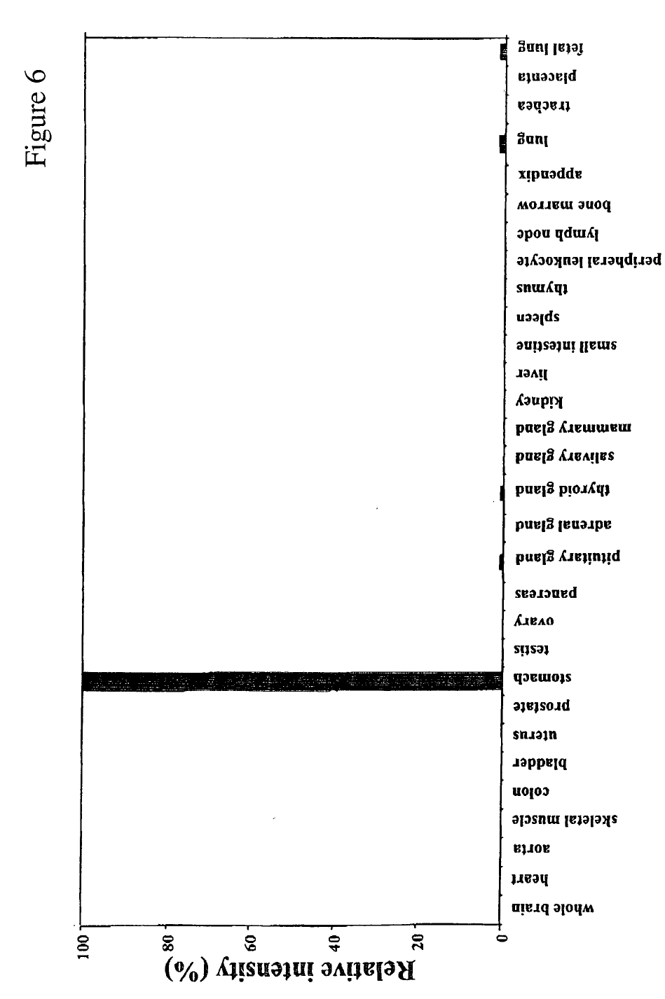

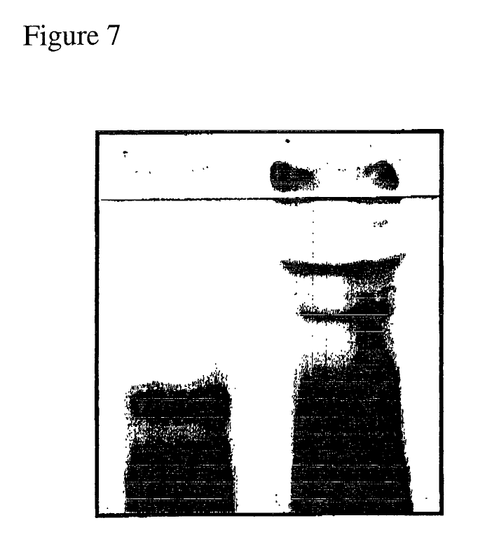

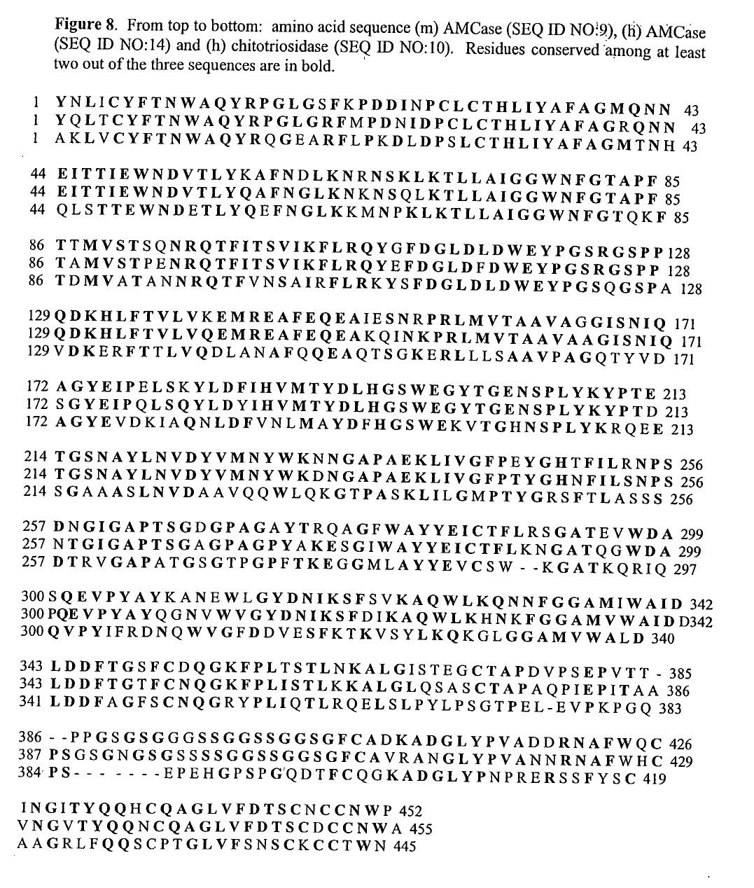

Reverse transcription-polymerase chain reaction (RT-PCR) fragments were generated from mouse lung total RNA using degenerate oligonucleotides, as described (Boot et al 1995). Obtained fragments were cloned in pGEM-T (Promega, Madison, Wis., USA), sequenced and compared with the amino acid sequence established by N-terminal protein sequencing. A comparison with the GenBank mouse EST (expressed sequence tag) database using the Basic local alignment search tool (BLAST) at NCBI (National Center for Biotechnology Information) showed that several EST clones matched the mouse chitinase cDNA sequence. For example ms33h09.y1 (GenBank Accession Number AI892792). This clone was obtained and sequenced. Anti-sense primers were generated complementary to the most 3′ region of the EST clone (A-tail primer: 5′-TTTTGGCTACCAATITTTATTGC-3′) and two internal anti-sense primers (MAS1: 5′-CAGCTACAGCAGCAGTAACCATC-3) and (MAS2: 5′-TTCAGGGATCTCATAGCCAGC-3′). The MAS1 and MAS2 primers were used to clone the most 5′ end of the mouse acidic chitinase cDNA using 5′ rapid amplification of cDNA ends (5′ RACE) and the Marathon-Ready mouse Lung cDNA kit (Clontech) according to the instructions of the manufacturer. To obtain the complete coding sequence a 5′ sense primer was generated (MS15′-CGATGGCCAAGCTACTTCTCGT-3′). The total cDNA sequence was subsequently generated using MS1 and the A-tail primer. The fragments of two independent PCR's were cloned into pGEM-T (Promega) and the nucleotide sequence of two independent clones from each PCR were sequenced from both strands by the procedure of Sanger using fluorescent nucleotides on an Applied Biosystems (ABI) 377A automated DNA sequencer following ABI protocols. The mouse AMCase protein shows considerable sequence homology to human chitotriosidase. Comparison of the amino acid sequence of both mature proteins revealed an identity of 52% and a similarity of 60%. Like the human chitotriosidase, the mouse enzyme is predicted to contain an N-terminal catalytic domain of about 39 kDa, a hinge region and a C-terminal chitin binding domain (FIG. 2). The mouse AMCase, like chitotriosidase, is predicted to lack N-linked oligosaccharides, explaining the observed absence of binding to Concanavalin A (data not shown). The apparent molecular masses of identically produced recombinant human chitotriosidase and recombinant mouse AMCase are comparable when run on a SDS-PAGE gel under reducing conditions. However, under non-reducing conditions, the mouse AMCase migrates significantly slower than the human chitotriosidase (FIG. 4A). Upon gelelectrophoresis (under non-reducing conditions) in polyacrylamide gels containing glycolchitin, followed by regeneration of active enzyme and detection of the local digestion of glycolchitin using Calcofluor staining, the mouse AMCase migrates slightly faster than human chitotriosidase (FIG. 4B).

Human AMCase

It was investigated whether such an acidic chitinase is also present in man. Screening the human EST database at the NCBI with the mouse acidic chitinase cDNA, revealed the presence of a highly homologous human EST clone (oq35c04.s1, GenBank Accession Number, AA976880). As mouse AMCase activity was shown in the stomach, the full length human AMCase cDNA was cloned using human stomach total RNA (Clontech) for the RT-PCR with the same degenerate primers as for the mouse AMCase. A human Marathon-Ready Lung cDNA was used to clone the most 5′ end of the cDNA by 5′ RACE using the following primers: HAS2 (5′-TCTGACAGCACAGAATCCACTGCC-3′) and HAS3-A-tail (5′-TTGACTGCTGATTTTATTGCAG-3′). The total cDNA sequence was subsequently generated using HS1 (5′-GCTTCCAGTCTGGTGGTGAAT-3) and HAS3-Atail. The fragments of two independent PCR's were cloned in pGEM-T (Promega) and sequenced as described above (FIG. 8A).

Screening the GenBank database using the full-length human cDNA revealed that it was almost identical to TSA1902-L (GenBank Accession Number AB025008) and TSA1902-S (GenBank Accession Number AB025009) from a lung cDNA library described by Saito et al. (Saito et al 1999). These two sequences are most probably splice variants of the human acidic chitinase mRNA. Only expression of full length human AMCase cDNA in COS-1 cells led to the production of a protein with chitinolytic activity (data not shown). Sequence comparison of the human acidic chitinase and the mouse acidic chitinase revealed an 82% identity and a similarity of 86% (FIG. 8B).

The demonstration by Saito et al. that the gene encoding TSA1902 is located on chromosome 1p13 Saito et al 1999) indicates that mammals contain indeed at least two discrete genes that encode functional chitinases, being chitotriosidase (locus 1q32) and AMCase (locus 1p13).

### Example 2

### Tissue Expression of Human and Mouse AMCase RNA

Another major difference between human chitotriosidase and the mouse AMCase is revealed by comparison of RNA expression patterns. Total RNA was isolated using RNAzol B (Biosolve, Barneveld, The Netherlands) according to the instructions of the manufacturer. Northern blots, using 15 □g of total RNA, were performed as described (Boot et al 1995). Mouse RNA Master Blots (Clontech, Palo Alto, Calif., USA), were used to examine the tissue distribution of transcripts according to the instructions of the manufacturer. The following probes were used: the full length mouse acidic chitinase cDNA and glyceraldehyde-3-phosphate dehydrogenase (GAPDH) as control. Radio-labeling and hybridization was conducted as described previously (Boot et al 1995). Quantification of radioactivity was performed using a phosphor imager (Storm phosphor imager, Molecular Dynamics, Sunnyvale, Calif., USA).

Whereas human chitotriosidase mRNA is mainly found in lymph node, bone marrow and lung, the mouse AMCase mRNA is predominantly found in, of the screened tissues, stomach, submaxillary gland and, at a lower level, in the lung (FIG. 6). Surprisingly, no mouse acidic chitinase mRNA could be detected in the small intestine. This can be explained by absence of mRNA, or by mRNA levels in the sample that were too low for detection with the technique used. These results suggest that the protein in the intestine is probably derived from the upper parts of the gastrointestinal tract, such as the stomach. In rat tissues a comparable acidic chitinase was observed. Our findings indicate that the acidic chitinase in rodents is distinct from human chitotriosidase. The discrete enzyme is therefore referred to as acidic SO mammalian chitinase or AMCase.

Next, the tissue distribution of this human mRNA was examined. Total RNA was isolated as decribed above and human RNA Master Blots (Clontech, Palo Alto, Calif., USA), were probed with the human EST clone oq36c04.s1 (GenBank Accession Number AA976830) and glyceraldehyde-3-phosphate dehydrogenase (GAPDH) as control to examine the tissue distribution as described above. The expression pattern of the human AMCase mRNA is similar to the expression pattern of the mouse acidic chitinase (FIGS. 6,7), being highly expressed in the stomach and at a lower level in the lung, as far as the RNA master blots are representative for these tissues. Expression in additional tissues that were not tested cannot be excluded.

### Example 3

### Degradation of Chitin by AMCase

Several different assays revealed that the mouse acidic chitinase is able to degrade chitin, and therefore has to be considered to be a true chitinase. Crab shell chitin (Poly-[1-4]-β-D-N-acetylglucosamine, Sigma) was used as a natural substrate to determine chitinase activity as described (Renkema et al 1997). The chitin fragments were analyzed by fluorophore assisted carbohydrate electrophoresis (FACE) as described by Jackson (Jackson 1990). FACE analysis revealed that recombinant mouse chitinase, like chitotriosidase, releases mainly chitobioside fragments from chitin (FIG. 8). Chitinase enzyme activity was determined in another assay with the fluorogenic substrates 4MU-chitobiose (4-methylumbelliferyl β-D-N,N′-diacetylchitobiose, Sigma, St Louis, USA) and 4MU-chitotriose (4-methylumbelliferyl β-D-N,N′,N″-triacetylchitotriose, Sigma). Assay mixtures contained 0.027 mM substrate and 1 mg/ml of bovine serum albumin (BSA) in McIlvaine buffer (100 mM citric acid, 200 mM sodium phosphate) at the indicated pH. The standard enzyme activity assay for human chitotriosidase with 4MU-chitotriose substrate was performed at pH 5.2, as previously described (Hollak et al 1994). The standard AMCase enzyme activity assays with 4MU-chitobiose substrate were performed at pH 4.5. Like chitotriosidase and some other non-mammalian chitinases, the mouse acidic chitinase activity in this assay is strongly inhibited (IC₅₀ of 0.4 □M) by the competitive chitinase inhibitor allosamidin (Milewski et al 1992, Dickinson et al 1989, McNab and Glover 1991). Measurements of chitin formation during regeneration of fungal spheroplasts was performed as described by Hector and Braun (Hector and Braun 1986). Briefly, spheroplasts were prepared from the Candida albicans strain CAi-4 (ura3), grown overnight in YPD medium (1% yeast extract, 2% peptone, 2% glucose) at 28° C. Cells were concentrated by centrifugation and incubated with 2.5 mg/ml zymolyase (100T, ICN Immuno Biologicals, Costa Mesa, Calif., USA) in buffer containing 50 mM sodium phosphate pH 7.5, 1.2 M sorbitol and 27 mM β-mercaptoethanol for 60 minutes at 37° C. After extensive washing, spheroplasts were allowed to regenerate in 96 wells microtiter plates in regeneration buffer (0.25% (w/v) MES buffer pH 6.7, containing 0.17% (w/v) Yeast Nitrogen Base (without amino acids and ammonium sulfate, Sigma), 0.15% (w/v) ammonium sulfate, 2% (w/v) glucose, 1.2 M sorbitol, 20 μg/ml uridine) at 87° C. Chitinase enzyme preparations were added in 3 μg/ml. After a 2 hour incubation, 50 μl of 300 μg/ml Calcofluor white (Sigma) in 10 mM sodium phosphate buffer pH 7.5 containing 1.2 M sorbitol was added. After 5 minutes the plates were washed with buffer only and fluorescence was determined using a LS 50 Perkin Elmer fluorimeter (excitation 405 nm, emission 450 nm).

In addition, the mouse acidic chitinase and chitotriosidase were both able to digest chitin in the cell wall of regenerating spheroplasts of Candida albicans. The chitin content of the cell wall was determined with the Calcofluor white stain When regenerating cells were incubated for 2 hours with 3 μg per ml recombinant chitotriosidase or 3 μg per ml recombinant mouse acidic chitinase the chitin content was reduced by 27% and 33%, respectively. Concomitant presence of allosamidin during the incubation completely abolished the effect of both recombinant chitinases. Finally, Incubation of hyphae of Candida albicans with recombinant 50 kDa or 89 kDa AMCase as described by (Boot et al 1995) results in selective lysis of the growing tip, showing a fungistatic effect of AMCase similar to the effect of chitotriosidase described by (Boot et al 1995).

### Example 4

### pH Optimum of Chitinase Activity AMCase

A striking difference between chitotriosidase and AMCase is their behavior at acidic pH.

The pH dependence of chitinase activity of AMCase was determined by incubating purified enzyme at different pH (McIlvaie buffer range 1.7-8.5) with 4-methylumbelliferyl substrates. Release of the fluorescent4-metylumbelliferone was monitored fluorometrically (excitation 445 nm and emission 360 nm). The results are presented in FIG. 5A. The mouse acidic chitinase shows a pronounced pH optimum at pH 2.8 and a less pronounced optimum at more neutral pH (pH 4-7). Chitotriosidase, however, shows only a broad pH optimum (see FIG. 5A) and is completely inactivated by pre-incubation at low pH (see FIG. 5B). In the presence of 0.5% (w/v) trichloroacetic acid (TCA) 58% of chitotriosidase is precipitated while under similar circumstances the mouse acidic chitinase remains in solution. At 2.5% (w/v) TCA all chitotriosidase precipitates while 26% of mouse acidic chitinase remains unprecipitated (FIG. 5C).

### Example 5

### Degradation of mammalian mucin by AMCase

The effect of AMCase on glycoproteins was studied by evaluating the effect of AMCase on mucin, a glycoprotein expressed on mucosal surfaces in the airways and gastrointestinal tract. ‘Bovine submaxillary gland mucin (Sigma) 125 μg was dissolved in 40 μl 0.05M NaAc pH 5.0 in the presence or absence of 500 ng mouse AMCase. After overnight incubation at 37’, the mucin was electrophorized on an SDS-PAGE gel (7.5% homogeneous, followed by silver staining). As is shown in FIG. 9 the mucin was degraded by treatment with the AMCase. This shows that AMCase is an endogenous regulator of mucus viscosity that can be employed in diseases in which overproduction or high viscosity of mucus is involved. Similar experiments may be performed using human AMCase to demonstrate its mucinolytic activity.

Experiments can be performed as described by Puchelle et al (1996) to demonstrate that AMCase is capable of decreasing the viscosity of cystic fibrosis sputum and sputum derived from chronic obstructive pulmonary disease patients.

### Example 6

### pH Optimum of Mucin Hydrolizing Activity of AMCase

The pH dependence of the mucin hydrolyzing activity of AMCase is determined by incubating purified enzyme at different pH (for example in McIlvaine buffer range 1.7-8.5) with mammalian mucin (see example 5).

### Example 7

### Generation of Antibodies to AMCase

Polyclonal antiserum against AMCase is raised by immunization of rabbits recombinant human AMCase (eg 4 injections/animal; 20-200 μg/injection). After the final booster the animals are bled to determine the titer of the polyclonal antiserum.

To obtain monoclonal antibodies 6-8 wk old Balb/c mice are immunized with recombinant human AMCase (for example 4 times with 2 wk intervals with 10-100 μg/injection dissolved in Freunds complete adjuvans for the first injection, and Freunds incomplete adjuvans for subsequent immunizations). Splenocytes are isolated and fused with a fusion cell line such as Sp2/0 myeloma cells, followed by limiting dilution. Growing clones are screened using for example an enzyme-linked immunosorbant assay (ELISA). Therefore 96 wells plates are coated with recombinant human AMCase or with a control protein. The culture supernatant is added, followed by washing and addition of a labeled anti-mouse antibody for detection. After limited dilution cloning of AMCase-specific antibody producing hybridomas stable hybridomas are obtained. From each clone cell supernatant is collected and by affinity chromatography using protein A sepharose columns (Pharmacia, Uppsala, Sweden) monoclonal antibodies are purified.

### Example 8

### A Quantitative Assay to Detect AMCase

Using the AMCase-specific antibodies of Example 7 and recombinant human AMCase, a quantitative assay for the detection of AMCase is set up. The assay is used to quantify AMCase. In addition, the assay is able to differentiate between chitotriosidase and AMCase.

### Example 9

### Anti-Fungal Activity of AMCase in Animal Models

Allergic bronchopulmonary aspergillosis is a complication that is often seen in CF and asthma patients. As AMCase has an effect on mucus as well as Aspergillus, proof of concept is obtained in a lung infection model with Aspergillus, in which mouse AMCase is administered intratracheally.

### Example 10

### Mucolytic Activity of AMCase in a Mouse Model for Cystic Fibrosis

CFTR-knockout mice are currently the only relevant disease model for human CF. The mice have the same genetic defect as CF patients. CFTR-knockout mice display many characteristics of intestinal disease in CF, but fail to develop respiratory infections or other signs of overt lung disease. To prevent massive mortality in the CFTR-knockout mice by intestinal obstruction animals need to be fed a liquid diet during the weaning period. Interestingly, CFTR-knockout mice that also are deficient for MUC1 have a much better survival on a solid diet, indicating a important role for mucus formation in the intestine of the CFTR-knockout mice. Clearly the model has its limitations for studying the effect of AMCase, however it is a good model to study the effect of AMCase on gastrointestinal disease seen in CF.

### Example 11

### Treatment of Mucin-Associated Human Diseases

Treatment of diseases that are typically associated with the undue, inappropriate or excessive production, or the insufficient removal of mucous are treated or ameliorated with mucinase. These diseases include but are not limited to COPD and CF. In the case of CF the indication for use of the mucinase is for both the digestive tract as well as the pulmonary signs and symptoms of excessive amounts of mucous present. Administration routes of the mucinase are such that the highest effective dose can be administered to the relevant anatomical area indicated for mucinolytic treatment.

### Example 12

### Treatment of Topical Infections by Chitin-Containing Pathogens

A variety and myriad number of dermatological and pulmonary tract fungal infections are deemed to be indications for treatment with a mucinase of the invention.

These include but are not limited to the Dermatophytoses (Tinea's), Candidiasis, Aspergillosis, Mucormycosis and Pneumocystis carinii. Patients presenting with dermatological manisfestations of fungal disease can be treated by the regular application of a topical formulation of a mucinase of the invention appropriate for the relevant anatomical region infected.

### REFERENCES

Almeida I. C., M. A. J. Ferguson, S. Schenkman & L. R. Travassos: Lytic anti-a-galactosyl antibodies from patients with chronic chagas' disease recognize novel O-linked oligosaccharides on mucin-like glycosyl-phosphatidylinositol-anchored glycoproteins of Trypanosoma cruzi. Biochem J 304, 793-802 (1994)

Araujo, A. C., Souto-Padron, T., and de Souza, W. J.: Cytochemical localization of carbohydrate residues in microfilariae of wuchereria bancrofti and brugia malayi. (1998) Histochem. Cytochem. 41(4) 571-578

Argueso P, Herreras J M, Calonge M, Citores L, Pastor J C, Girbes T. Analysis of human ocular mucus:effects of neuraminidase and chitinase enzymes. Cornea 17 200-207 1998.

Barnes D. A., A. Bonnin, J. X. Huang, L. Gousset, J. Wu, W. J. Gut, P. Doyle, J. F. Dubremetz, H. Ward & C. Petersen: A novel multi-domain mucin-like glycoprotein of Cryptosporidium parvum mediates invasion. Mol Biochem Parasitol 96, 93-110 (1998)

Boot, R. G., Renkema, G. H., Strijland, A., van Zonneveld, A. J., and Aerts, J. M. F. G.: Cloning of a cDNA encoding chitotriosidase, a human chitinase produced by macrophages. (1995) J. Biol. Chem. 270, 26252-26256

Boot, R. G., Renkema, G. H., Strijland, A., van Zonneveld, A. J., and Aerts, J. M. F. G.: Cloning of a cDNA encoding chitotriosidase, a human chitinase produced by macrophages. (1995) J. Biol. Chem. 270, 26252-26256

Boot R. G., E. F. C. Blommaart, E. Swart, K. Ghauharali-van der Vlught, N. Bijl, C. Moe, A. Place, and J. M. F. G. Aerts: Identification of a novel acidic mammalian chitinase distinct from chitotriosidase (2001) J. Biol. Chem. 276, 6770-6778

Buhrmester, C C. (1983). A study of the hydrogen-ion concentration, nitrogen content and viscosity of nasal secretions. Ann. Otol. Rhinol. Laryngol. 42:778-788.

Carol A. Vasconcellos, Philip G. Allen, Mary Ellen Wohl, Jeffrey M. Drazen, Paul A. Janmey, Thomas P.

Stossel: Reduction in viscosity of cystic fibrosis sputum in vitro by gelsolin. Science 263, 969-971 (1994)

Carraway K. L., S. A. Price-Schiavi, M. Komatsu, N. Idris, A. Perez, P. Li, S. Jepson, X. Zhu, M. E. Carvajal & C. A. Carraway: Multiple facets of sialomucin complex/MUC4, a membrane mucin and erbb2 ligand, in tumors and tissues (Y2K update). (2000) Front Biosci 5, D95-D107

Celli B R: Pulmonary rehabilitation in patient with COPD. Am. J. Respir. Crit Care Med. 1995 152(3):861-4

Chernick W S, Barbero G J. Composition of tracheobronchial secretions in cystic fibrosis of the pancreas and bronchiectasis. Pediatrics. 1959: 24: 739-745

Choi I, C Aalkjaer, E L Boulpaep, and W F Boron. (2000). An electroneutral sodium/bicarbonate cotransporter NBCn1 and associated sodium channel. Nature 405:571-575.

Cockrill B A, and Hales Calif. Allergic Bronchoalveolar Aspergillosis. Ann. Rev. Med. 1999. 50:303-316.

Debono M., and Gordee R S.: Antibiotics that inhibit fungal cell wall development. Ann rev Microbiology 1994 48, 471-497

Dickinson, K., Keer, V., Hitchcock, C. A. and Adams, D. J.: Chitinase activity from candida albicans and its inhibition by allosamidin. (1989) J. Gen. Microbiol. 135, 1417-142

Escott, G. M., and Adams, D. J.: Chitinase activity in human serum and leukocytes. (1995) Infect. Immun. 63(12), 4770-4773

Flach, J., Pilet, P. E., and Jolles, P.: Whats new in chitinase research? (1992) Experientia 48, 701-716

Fuchs H J, Borowitz D S, Christiansen D H, Morris E M, Nash M L, Ramsey B W, Rosenstein B J, Smith A L, Wohl M E: Effect of aerosolized recombinant human Dnase on exacerbations of respiratory symptoms and on pulmonary function in patients with cystic fibrosis. The Pulmozyme Study Group. New England Journal of Medicine Septembe 1994, 331(10):637-642

Fuhrman, J. A., and Piessens, W. F.: Chitin synthesis and sheath morphogenesis in bruglia malayi microfilariae (1985) Mol. Biochem. Parasitol. 17, 93-104

Gendler S. J. & A. P. Spicer: Epithelial mucin genes. Annu Rev Physiol 57, 607-634 (1995)

Gum J. R. J.: Human mucin glycoproteins: varied structures predict diverse properties and specific functions. (1995) Biochem Soc Trans 23, 795-799

Gum J. R., J. W. Hicks, D. M. Swallow, R. L. Lagace, J. C. Byrd, D. T. Lamport, B. Siddiki, & Y. S. Kim: Molecular cloning of cDNAs derived from a novel human intestinal mucin gene. (1990) Biochem Biophys Res Commun 171, 407-415

Hector, R. F., and Braun, P. C.: A 96 well epifluorescence assay for rapid assessment of compounds inhibitory to candida spp. (1986) J. Clin. Microbiol. 24, 620-624

Hollak, C. E. M., van Weely, S., van Oers, M. H. J., and Aerts, J. M. F. G.: Markes elevation of plasma Chitotriosidase activity. A novel hallmark of Gaucher disease. (1994) J. Clin. Invest. 93, 1288-1292

Hollak, C. E. M., van Weely, S., van Oers, M. H. J., and Aerts, J. M. F. G.: Markes elevation of plasma Chitotriosidase activity. A novel hallmark of Gaucher disease. (1994) J. Clin. Invest. 98, 1288-1292

Hudson M. J. H., G. W. H. Stamp, M. A. Hollingworth, M. Pignatelli & E. N. Lalani. MUC1 expressed in PanC1 cells decreases adhesion to type1 collagen but increases contraction in collagen lattices. (1996) Am J Pathol 148, 951-960

Hunt J F, K. Fang, R Malik, A Snyder, N malhotra, T A E Platts-Mills, and B Gaston. (2000) Endogenous airway acidification. Implications for asthma pathophysiology. Am. J. Resp. Crit. Care Med. 161:694-699.

Ilg T., E. Handman, N. Ken, Y. D. Stierhof & A. Bacic: Mucin-like proteophosphoglycans from the protozoan parasite Leishmania. (1999). Trends Glycosci Glycotech 11, 53-71

Jackson, P.: The use of polyacrylamide-gel electrophoresis for the high-resolution separation of reducing saccharides labeled with the fluorophore 8-amino naphthalene-1,3,6-trisulphonic acid. Detection of picomolar quantities by an imaging system based on a cooled charge-coupled device. (1990) Biochem. J. 270, 705-713

Jeffery P. K., D. Gaillard & S. Moret: Human airway secretory cells during development and in mature airway epithelium. (1992) Eur Respir J 5, 93-104

N. Jentoft: Why are proteins O-glycosylated? (1990) Trends Biochem Sci 15, 291-294

Kim J S, Hackley G H, Okamoto X, Rubin B K. (2001) Sputum processing for evaluation of inflammatory mediators. Pediatr Pulmonol 32:152-8

Kastrup E K, et al. (eds) Respiratory inhalant products. In Drug Facts and Comparisons, (1998). St. Louis, Facts and Comparisons. pp 1141-1163.

Lan M. S., S. K. Batra, W. N. Qi, R. S. Metzgar & M. A. Hollingsworth: Cloning and sequencing of a human pancreatic tumor mucin cDNA. (1990) J Biol Chem 265, 15294-15299

Komatsu M., L. Tatum, N. H. Altman, C. A. Carothers, K. Carraway & L. Carraway: Potentiation of metastasis by cell surface sialomucin complex (rat MUC4), a multifunctional anti-adhesive glycoprotein. (2000) Int J Cancer 87, 480-486

Lowry, O. H., Rosebrough, N. J., Farr, A. L., and Randall, R. J. (1951) J. Biol. Chem. 193.265-275

McNab, R., and Glover, L. A.: Inhibition of neurospora crassa cytosolic chitinase by allosamidin (1991) FEMS Microbiol. Lett. 82, 79-82

Milewski, S., O'Donnel, R. W., and Gooday, G. W.: Chemical modification studies of the active center of candida albicans chitinase and its inhibition by allosamidin (1992) J. Gen. Microbiol. 188, 2545-2550

Moniaux N., S. Nollet, N. Porchet, P. Degand, A. Laine & J. P. Aubert: Complete sequence of the human mucin MUC4: a putative cell membrane-associated mucin. (1999) Biochem J 338, 325-333

Neville, A. C., Parry, D. A., Woodhead-Galloway, J.: The chitin crystallite in arthropod cultide (1976) J Cell Science, 21, 73-82

Plant P K, J L Owen, M W Elliott. (2000). One year period prevalence study of respiratory acidosis in acute excacerbations of COPD: implications for the provision of non-invasive ventilation and oxygen administration. Thorax 55:550-554.

Potter J, Matthews L W, Lemm J. et al. The composition of pulmonary secretions from patients with and without cystic fibrosis. Am. J. Dis. Child (1960). 100:493-495

Puchelle E., J-M. Zahm, S. de Bentzmann, C. Grosskopf, S. Shak, D. Mougel, J-M. Polu: Effects of rhDNase on purulent airway secretions in chronic bronchitis. Eur Respir J, (1996), 9,765-769

Renkema, G. H., Boot, R. G., Muijsers, A. O., Donker-Koopman, W. E., and Aerts, J. M. F. G.: Purification and characterisation of human chitotriosidase, a novel member of the chitinase family proteins. (1995) J. Biol. Chem. 270, 2198-2202

Renkema, G. H., Boot, R. G., Strijland, A. Donker-koopman, W. E., van den Berg, M., Muijsers, A. O., and Aerts, J. M. F. G.: Synthesis, sorting, and processing into distinct isoforms of human macrophage chitotriosidase. (1997) Eur. J. Biochem. 244, 279-28511.

Riordan J R, Rommens J M, Kerem B, Alon N, Rozmahel R, Grzelczak Z, Zielenski J, Lok S, Plavsic N, Chou J L: Identification of the cystic fibrosis gene: cloning and characterization of complementary DNA. Science. (1989)245:1066-73.

Saito A., K. Ozaki, T. Fujiwara, Y. Nakamura, A. Tanigami: Isolation and mapping of a human lung-specific gene, TSA1902, encoding a novel chitinase family member. Gene 239 (1999) 325-331

Schenkman S., M. A. J. Ferguson, N. Heise, M. L. C. Almeida, R. A. Mortara & N. Yoshida: Mucin-like glycoproteins linked to the membrane by glycosylphosphatidylinositol anchor are the major acceptors of sialic acid in a reaction catalyzed by trans-sialidase in metacyclic forms of Trypanosoma cruzi. (1993) Mol Biochem Parasitol 59, 293-304

Shahabuddin, M., and Kaslow, D. C.: Plasmodium: parasite chitinase and its role in maloria transmission (1994) Exp. Parasit. 79, 85-88

Shak S, Capon D J, Hellmiss R, Marsters S A, and Baker C L.: Recombinant human Dnase I reduces the viscosity of cystic fibrosis sputum. (1990) Proc. Natl. Acad. Sci. USA 87, 9188-9192

Shak S: Aerosolized recombinant human Dnase I for the treatment of cystic fibrosis. Chest (1995) 107:65s-70s

Shankar V., P. Pichan, R. L. J. Eddy, V. Tonk, N. Nowak, S. N. Sait, T. B. Shows, R. E. Schultz, G. Gotway, R. C. Elkins, M. S. Gilmore & G. P. Sachdev: Chromosomal localization of a human mucin gene (MUC8) and cloning of the cDNA corresponding to the carboxy terminus. (1997) Am J Respir Cell Mol Biol 16, 232-241

Shimahara, K., & Y. Takiguchi. (1988) Preparation of crustacean chitin. Methods Enzymol. 161:417-423.

Sternberg L. R., J. C. Byrd, C. K. Yunker, S. Dudas, V. K. Hoon & R. S. Bresalier: Liver colonization by human colon cancer cells is reduced by antisense inhibition of MUC2 mucin synthesis. (1999) Gastroenterol. 116, 363-371

Strong W. B., J. Gut & R. G. Nelson: Cloning and sequence analysis of a highly polymorphic Cryptosporidium parvum gene encoding a 60-kilodalton glycoprotein and characterization of its 15- and 45-kilodalton zoite surface antigen products. Infect Immun (2000) 68, 4117-4134

Strous G. J & J. Dekker: Mucin-type glycoproteins. (1992) Crit Rev Biochem Mol Biol 27, 57-92

Suzuki M, Morimatsu M, Yamashita T, Iwanaga T, Syuto B. (2001) A novel serum chitinase that is expressed in bovine liver. FEBS letter 506:127-130

Trudel, J., and Asselin, A.: Detection of chitinase activity after polyacrylamide gel electrophoresis. (1989) Anal. Biochem. 178, 362-366

Williams S. J., M. A. McGuckin, D. C. Gotley, H. J. Eyre, G. R. Sutherland & T. M. Antalis: Two novel mucin genes down-regulated in colorectal cancer identified by differential display. (1999) Cancer Res 59, 4088-4089

Zahm J M, Girod de Bentzmann, Deneuville E, Perrot-Minnot C, D Pennaforte F, Roussey M, Shak S, Puchelle E: Dose- dependent in vitro effect of recombinant human Dna rheological and transport properties of cystic fibrosis respi mucus. Eur Respir J (1995) 8:381-386

What is claimed is:

1. A recombinant and/or substantially isolated or purified mammalian mucinase, or a modified form thereof having a substantially similar mucin-hydrolyzing activity.

2. A recombinant and/or substantially isolated or purified mucinase, said mucinase being a mucinase having an amino acid sequence essentially corresponding to the amino acid sequence shown in FIG. 8, or a modified form of said mucinase having a substantially similar mucin hydrolyzing activity.

3. The mucinase of claim 1 or claim 2, produced by a host or host cell and isolated from said host, host cell or medium in which said host cell is cultured.

4. The mucinase of claim 3, wherein the amino acid sequence of said mucinase is encoded by a nucleotide sequence essentially corresponding to the nucleotide sequence shown in FIG. 8.

5. A pharmaceutical composition comprising an effective amount of the mucinase of any one of the claims 1 to 4 and a pharmaceutically acceptable carrier or diluent.

6. A pharmaceutical composition for treatment or prophylaxis of a subject against a disease in which mucus is involved, said pharmaceutical composition comprising:
a therapeutically or prophylactically effective amount of the mucinase of any one of the claims 1 to 4, and
a pharmaceutically acceptable carrier or diluent.

7. The pharmaceutical composition of claim 6, which further comprises a therapeutically or prophylactically effective amount of a second pharmaceutical composition, such as human DNAse1, a mucolytic, an antibiotic, a pancreatic enzyme supplement, an antifungal drug, an antihistamine, a bronchodilator, a leukotrien inhibitor, and/or a corticosteroid.

8. A composition comprising the mucinase of any one of the claims 1 to 4 and a carrier or diluent.

9. The composition of claim 8, which is a cosmetic, dental or food product.

10. A method of therapeutic or prophylactic treatment of a subject against a disease in which mucus is involved, said method comprising administering to the subject the pharmaceutical composition of claim 5, claim 6, or claim 7.

11. A method for preparing a mammalian mucinase, or a modified form thereof having a substantially similar mucin-hydrolyzing activity, said method comprising:
growing a host or host cell capable of producing said mucinase or modified form thereof and
isolating the mucinase produced from said host or host cell or from medium in which said host cell is cultured.

12. The method according to claim 11, wherein said mucinase comprises an amino acid sequence essentially corresponding to the amino acid sequence shown in FIG. 8, or a modified form of said mucinase having a substantially similar mucine-hydrolyzing activity.

13. The method according to claim 11 or 12, wherein said host or host cell comprises a genetically engineered host or host cell.

14. The method according to any one of the claims 11 to 13, wherein the amino acid sequence of said mucinase is encoded by a nucleotide sequence essentially corresponding to the nucleotide sequence shown in FIG. 8.

15. The mucinase of any one of the claims 1 to 4, further comprising
a chitin-hydrolyzing activity.

16. A pharmaceutical composition for therapeutic or prophylactic treatment of a subject against infection by a chitin-containing pathogen, said pharmaceutical composition comprising:
a therapeutically or prophylactically effective amount of the mucinase of claim 15 and
a pharmaceutically acceptable carrier or diluent.

17. A fusion protein comprising:
the mucinase of any one of the claims 1 to 4 or 15 and/or a functional part thereof, and
a protection moiety.

18. A composition comprising the mucinase of any one of claims 1 to 4 or 15 and a carrier or diluent.

19. The composition of claim 18, which is a medium for culturing cells.

20. The composition of claim 18, which is a medium for culturing human cells.

21. The composition of claim 18, which is a cosmetic, dental, or food product.

22. A method of therapeutic or prophylactic treatment of a subject against infection by a chitin-containing pathogen, said method comprising:
administering to the subject the pharmaceutical composition of claim 16.

23. A chitin-based article of manufacture comprising:
a chitin-hydrolyzing amount of the mucinase of claim 15.

24. The chitin-based article of manufacture of claim 23, wherein said article of manufacture is a drug-containing drug carrier or implant for controlled drug release.

25. The chitin-based article of manufacture of claim 23, wherein said article of manufacture is a transient functional implant.

26. An isolated host cell capable of producing a mammalian mucinase.

27. The isolated host cell of claim 26 wherein said host cell is capable of producing a mucinase having an amino acid sequence essentially corresponding to the amino acid sequence shown in FIG. 8, or a modified form of said mucinase having a substantially similar mucine-hydrolyzing activity.

28. The host cell of claim 26 or claim 27, wherein said host cell is genetically engineered to produce an altered amount of mammalian mucinase.

29. A recombinant nucleic acid comprising a nucleotide sequence encoding, or complementary to a nucleotide sequence encoding, an expressable mammalian mucinase.

30. The recombinant nucleic acid of claim 29, wherein said mucinase comprises an amino acid sequence essentially corresponding to the amino acid sequence shown in FIG. 8.

31. The recombinant nucleic acid of claim 29 or claim 30, wherein said nucleotide sequence essentially corresponds to, or essentially is complementary to, the nucleic acid sequence shown in FIG. 8.

32. An oligonucleotide of at least about 8 nucleotides having a nucleotide sequence corresponding to, or complementary to, a nucleotide sequence shown in FIG. 8 and being capable of binding by hybridization under stringent hybridization conditions to nucleic acid coding for the mucinase of any one of the claims 1 to 4 or 15.

33. A peptide of at least about 8 amino acid residues having an amino acid sequence derived from the amino acid sequence shown in FIG. 8 and representing or mimicking an epitope of the mucinase of any one of the claims 1 to 4 or 15.

34. The peptide of claim 33 having an amino acid sequence corresponding to an amino acid sequence shown in FIG. 8 and having antigenicity.

35. An antibody capable of binding to the mucinase of any one of the claims 1 to 4 or 15.

36. The antibody of claim 35, wherein said antibody is a monoclonal antibody.

37. A diagnostic kit of the type having an antibody together with a component for detecting an antigen or an antibody, wherein the improvement comprises:
selecting the antibody to be the antibody of claim 35 or claim 36.

38. A diagnostic kit of the type having a peptide together with a component for detecting an antigen or an antibody, wherein the improvement comprises:
selecting the peptide to be the peptide of claim 33 or claim 34.

39. A diagnostic kit of the type having an oligonucleotide together with a component for detecting a nucleic acid, wherein the improvement comprises:
selecting the oligonucleotide to be the oligonucleotide of claim 32.

40. A diagnostic kit comprising the recombinant nucleic acid of any one of claims 29 to 31 and a conventional component of diagnostic kits for detecting a nucleic acid.

41. A diagnostic kit comprising a diagnostically effective amount of the mucinase of any one of the claims 1 to 4 or 15 and a conventional component of diagnostic kits for detecting an antigen or antibody.

42. A method of decomposing mucin, said method comprising:
contacting said mucin with the mucinase of any one of the claims 1 to 4 or 15 under mucin hydrolyzing conditions.

43. A method of decomposing chitin comprising contacting said chitin with the mucinase of claim 15 under chitin-hydrolyzing conditions.

44. The method of claim 10 wherein said disease is selected from the group consisting of cystic fibrosis, chronic obstructive pulmonary disease, asthma, bronchitis, tuberculosis, a mucin producing tumor, and infection by a protozoan parasite.
